(19) United States
(12) Patent Application Publication
(10) Pub. No.: US 2011/0033862 A1
(43) Pub. Date: Feb. 10, 2011

(54) METHODS FOR CELL GENOTYPING

(75) Inventors: Matthew Rabinowitz, Portola Valley, CA (US); David S. Johnson, San Francisco, CA (US); Johan Baner, Palo Alto, CA (US); Zachary Demko, Somerville, MA (US); Cengiz Cinnioglu, Sunnyvale, CA (US)

Correspondence Address: GREENBERG TRAURIG, LLP, ONE INTERNATIONAL PLACE, 20th FL, ATTN: PATENT ADMINISTRATOR, BOSTON, MA, 02110, US

(73) Assignee: Gene Security Network, Inc.

(21) Appl. No.: 12/918,445

(22) PCT Filed: Feb. 19, 2009
(86) PCT No.: PCT/US09/34506

§ 371 Date: Oct. 7, 2010

(51) Int. Cl. C12Q 1/68; C12P 19/34

(52) U.S. Cl. 435/6; 435/91.5

(57) ABSTRACT

Methods for cell genotyping are disclosed herein. A method for determining the genomic data of one or a small number of cells, or from fragmentary DNA, where a limited quantity of genetic data is available may include adding one or more targeted primers to a whole genome amplification of a cell, increasing the accuracy with which key alleles are measured in the context of a whole genome amplification. The genetic material from a single cell may be divided into fractions, each of which may be separately genotyped, allowing the reconstruction of the cells haplotype. The genetic material from a single cell may be divided into fractions, each of which may be separately genotyped, and the distribution of the various alleles in the different fractions may be used to determine the ploidy state of one or a plurality of chromosomes in the cell.

### FIELD

The embodiments disclosed herein relate generally to the field of acquiring and/or manipulating high fidelity genetic data for medically predictive purposes, and more particularly to a system which allows genetic data from a single or small number of cells to be measured with high accuracy, and for the genetic haplotypes and ploidy states to be determined.

### BACKGROUND

### Preimplantation Genetic Diagnosis

In 2006, across the globe, roughly 800,000 in vitro fertilization (IVF) cycles were run. Of the nearly 140,000 cycles run in the US, about 10,000 involved pre-implantation genetic diagnosis (PGD). Current PGD techniques are unregulated, expensive and can be unreliable: error rates for screening disease-linked loci or aneuploidy are on the order of 10%, each screening test costs roughly $5,000, and a couple is forced to choose between testing aneuploidy, which afflicts roughly 50% of IVF embryos, or screening for disease-linked loci on the single cell. There is a great need for an affordable technology that can reliably determine genetic data from a single cell in order to screen in parallel for aneuploidy, monogenic diseases such as Cystic Fibrosis, and susceptibility to complex disease phenotypes which have multiple genetic markers.

The process of PGD during IVF currently involves biopsy of embryos generated using assisted conception techniques. There are two potential sources of embryonic genetic material for PGD aneuploidy screening: a single blastomere from cleavage stage embryos (typically day 3 post-fertilization) and 4-10 tropechtoderm cells from blastocyst stage embryos (typically day 5 post-fertilization). Using cleavage stage single cell biopsy is the most common approach to PGD. Isolation of single cells from human embryos, while highly technical, is now routine in IVF clinics. Polar bodies, blastomeres, and tropechtoderm cells have been isolated with success. However, there is only a limited amount of time available for preimplantation testing—most clinics aim to transfer the embryos to the mother within 32 hours of biopsy. Consequently, diagnostic methods must be rapid as well as accurate. It is possible, though not preferred by most IVF clinics, to cryopreserve embryos after biopsy, in which case they can be transferred months or even years after they have been isolated.

To biopsy an embryo, it is usually transferred to a special cell culture medium and a hole is introduced into the zona pellucida using an acidic solution, laser, or mechanical technique. The technician then uses a biopsy pipette to remove a single blastomere with a visible nucleus in the case of day 3 embryos, or a small number of tropechtoderm cells in the case of day 5 embryos. Features of the DNA of the biopsied cell(s) can be measured using a variety of techniques. Since only a single copy of the DNA is available from one cell, direct measurements of the DNA of one or a small number of cells are error-prone, or noisy. There is a great need for a technique that can improve the accuracy of these genetic measurements using a small number of cells.

In cases where the goal is to diagnose a particular genetic mutation, the amplification of the DNA is typically carried out using a single primer, or set of primers, specifically designed to amplify a particular locus or small number of loci of interest. When only one primer or a small set of primers is used, the allele dropout (ADO) rate tends to be quite low. However, in the case where the goal is to measure the genotype of the cell at hundreds or thousands of loci, the amplification is typically done with a method such as whole genome amplification (WGA). In these cases, one or a set of generic primers (sometimes called universal primers or random primers) are typically used. In these methods, ADO rate at a given allele tends to be significantly higher than when a targeted primer is used. There is a need for a technique that can combine the ability of WGA to genotype a large number of alleles with a method that can provide genotyping of key alleles with the high level of accuracy possible when performing targeted amplification.

### MDA and WGA

Whole genome amplification is widely used when analyzing the genome of a single cell, and in theory, results in the amplification of all loci on the DNA. One major limiting factor in this process are ADOs, i.e. missing information about one or both alleles of a locus. The mechanisms of ADOs are largely unknown and may occur during lysis, amplification or analysis of single cells. Typical ADO rates from whole genome amplifications typically range from 20 to 60%. In addition, the identity of alleles that drop out are effectively random.

Prior to the development of whole genome amplification, the most widely used method for analyzing specific loci in single cells was polymerase chain reaction (PCR), which requires many cells per locus in order to compensate for uninformative DNA fragments. However, PCR is not useful for analyzing more than one loci per cell because multiple PCR primers cannot be used simultaneously. Additionally, non-random amplifications, e.g. multiplex PCRs, can suffer from sequence specific ADO due to high GC containing loci or non-optimal primer pairs.

An alternative method of analyzing loci in single cells is fluorescent in situ hybridization (FISH), but the method is basically limited to a few loci per cell. Whole genome amplification has evolved as a means to analyze several targets post-amplification. However, random amplifications, e.g. using multiple displacement amplification (MDA) kit or a WGA kit act on every target sequence, and thus still suffer from random ADO.

In some contexts, such as PGD, it may be desirable to measure a large number of alleles, wherein a subset of those alleles is of particular importance. It is desirable to measure a large number of alleles, and simultaneously maximize the accuracy of the genotyping of those targeted alleles. Unfortunately, no method has yet been shown to decrease the likelihood of a desired subset of alleles to drop out upon amplification and genotyping, as compared to the overall set of alleles, in the context of WGA.

For example, if a clinician wishes to make predictions regarding the genetic susceptibility of an embryo to one or more potential diseases, it is necessary to be able to determine the identity of those disease-linked alleles in the embryo in question. Simply measuring the disease-linked allele carries a risk of an allele miss-call, or ADO. There is an as yet unmet need for such a technology which combines the ability to measure a large number of alleles and also the ability to maximize the accuracy with which a specific subset of those alleles is measured.

One method used to avoid the problem of ADO at alleles of interest is to measure multiple polymorphic alleles that lie within a disease gene and/or closely flanking it. This can enable the deduction of the high-risk (i.e. mutation carrying) haplotypes that have been inherited by embryo, and can overcome some of the difficulties associated with particular markers being uninformative for a given family, or the problem of ADO measure of disease-linked alleles. The knowledge of sufficient flanking polymorphic alleles, measurable using a whole genome amplification technique, along with the knowledge of the haplotype can increase the ability to correctly deduce the identity of the target allele(s) if it was not measured correctly. While the use of flanking alleles, measurable when using whole genome amplification techniques, can be used to increase the accuracy with which key alleles are determined, there is an as yet unmet need for a technology that can combine the ability to measure a large number of flanking alleles and at the same time decrease the likelihood of an uninformative measurement at the target allele itself.

### Single Cell Haplotyping

Many methods exist for genotyping—revealing which alleles an individual carries at different genetic loci. A harder problem is haplotyping—determining which alleles lie on each of the two homologous chromosomes in a diploid individual. For instance, an individual may have the genotype AB/ab (heterozygous at each of loci A and B), but could carry haplotypes AB and ab or, conversely, Ab and aB. Conventional approaches to haplotyping require the use of several generations of cells to reconstruct haplotypes within a pedigree, or use statistical methods to estimate the prevalence of different haplotypes in a population. Several molecular haplotyping methods have been proposed, but have been limited to small numbers of loci, usually over short distances.

Determining the phase or haplotype of two or more genetic variants (single nucleotide polymorphisms, SNPs) has long been a challenging task due to technical issues when working with a few or single DNA strands. Among the problems are loss of material (e.g. attachment to equipment), sensitivity (e.g. not enough material), or integrity (DNA is fragile and breaks easily). Integrity plays an important part when setting up any haplotyping assay, since fragmentation may affect the distance between SNPs that can be analyzed, or generate false haplotypes if diploid fragments are switched.

Haplotype-based methods offer a powerful approach to disease gene mapping, based on the association between the causal mutations and the ancestral haplotypes on which they arose. The genome, human or other species, can be parsed objectively into haplotype blocks: sizable regions over which there is little evidence for historical recombination and within which only a few common haplotypes are observed. The boundaries of such blocks and specific haplotypes they contain are highly correlated across populations; these haplotype frameworks provide substantial statistical power in association studies of common genetic variation across each region, and facilitate comprehensive genetic association studies of human disease.

Quantitative traits such as drug responsiveness or disease susceptibility may be more strongly correlated with certain haplotypes than with certain genotypes, particularly where several polymorphic loci fall within a single gene. Hence, both the discovery of an association between a trait and a polymorphism, and the implications of this association for an individual, depend on knowledge of haplotypes. Haplotype structure is also important in understanding the evolution of a species and of populations within it, as haplotype blocks are shuffled in successive generations. The persistence of ancestral haplotypes can also be used to simplify genotyping experiments: the genotype at one locus may serve as a proxy for the genotypes of neighboring loci if they lie within the same conserved haplotype block.

Current methods for haplotyping are quite diverse and involve for example PCR, FISH, and rolling circle amplification (RCA). The common theme is to isolate individual DNA strands, for example in emulsion phases, on glass slides, or circularize them, followed by amplification and readout, or in the case of FISH, hybridization of fluorescently labeled oligonucleotides. Most methods work with very low concentrations of input DNA to avoid scoring or mix up of both alleles of a SNP, making them relatively inefficient and hard to optimize.

Cloning in hybridomas or in bacterial or yeast cells, or the natural occurrence of hydatidiform moles arising from a single haploid gamete, can also be used to isolate a single haplotype which can then be revealed by simple genotyping. Such approaches, however, are limited to the analysis of small numbers of loci over short distances (typically a few hundred base pairs). Other methods have been based on the analysis by PCR of single DNA molecules which, of course, represent single haplotypes. The most direct implementation of this strategy is the genotyping of single sperm, in which meiosis has done the job of isolating a single copy of each chromosome.

Other approaches rely upon the division of genetic matter into fractions to isolate (statistically) single DNA molecules, followed by the specific amplification and genotyping of two or more loci. However, this method is unable to measure haplotypes of more than about 20-30 kb in length, that do not involve more than a few loci, and the methods are often inefficient since only a few of the highly dilute samples which are genotyped will prove to contain informative molecules. A method, described by Wetmur (Wetmur et al., Nucl. Acids Res., 2005, 33(8), 2615-2619), uses linking emulsion PCR (LE-PCR), which enables formation of mini-chromosomes preserving phase information of two polymorphic loci, hence the haplotype. A drawback to all of these methods is that they require a large number of DNA copies (thousands to millions of cells) to produce accurate results. There is a great need for a technique that can accomplish the effective haplotyping of a cell by isolating individual haplotypes from a single cell, or a small number of cells.

The main problem in haplotyping is knowing whether one is looking at information from a single continuous DNA strand (implied chromosome), or fragmented DNA strands belonging to the same chromosome copy. For example, if two consecutive SNPs (located on the same chromosome) are measured in a given reaction, there is a risk that there was chromosomal breakage between them, and one of the measured SNPs is actually from a homologous chromosome as the other measured SNP, thus resulting in a false haplotype deduction. Any method that involves conditions that induce DNA strand breakage, either through chemical or mechanical means, will have some likelihood of such false haplotyping.

Examples in the prior art dealing with this problem include dilution of DNA obtained from bulk tissue or blood samples down to levels where it is statistically unlikely (however still possible) that two copies of the same sequence ended up in the same reaction (Konfortov et al., Nucl. Acids Res., 2007, 35(1), e6). Alternatively one can break up the entire reaction into several fractions with the hope that each fraction contains a unique chromosome (Wetmur). Note that these techniques do not solve the problem, they just make it less probable that the problem will have an impact on a given haplotype determination. A significant drawback to these methods is that they are done with bulk genetic material derived from many cells, well more than a thousand, and typically in the millions. These methods have not been applicable to DNA samples derived from one or a small number of cells. Another drawback to these methods is that they typically start with prepared genomic DNA from many cells, and the methods of preparation typically result in a greater degree of DNA fragmentation, especially purification steps, thereby limiting how far apart along the genome different SNPs can be phased. There exists a need for a technique that can determine the haplotypes by isolating different haplotypes present in the genetic material found in one or a small number of cells. There exists a need for a technique that can determine the haplotypes by isolating different haplotypes present in the cells of an individual that minimizes the possibility of false haplotyping due to strand breakage.

### Aneuploidy

Normal humans have two sets of 23 chromosomes in every diploid cell, with one set from each parent. Aneuploidy, the state of a cell with extra or missing chromosome(s), and uniparental disomy, the state of a cell with two of a given chromosome both of which originate from one parent, is believed to be responsible for a large percentage of failed implantations and miscarriages, and some genetic diseases. When only certain cells in an individual are aneuploid, the individual is said to exhibit mosaicism. Detection of chromosomal abnormalities can identify individuals or embryos with conditions such as Down syndrome, Klinefelter's syndrome, and Turner syndrome, among others, and potentially increase the chances of a successful pregnancy. Testing for chromosomal abnormalities is especially important as the age of a potential mother increases: between the ages of 35 and 40 it is estimated that between 40% and 50% of the embryos are abnormal, and above the age of 40, more than half of the embryos are like to be abnormal. The main cause of aneuploidy is nondisjunction during meiosis. Maternal nondisjunction constitutes 88% of all nondisjunction, of which 65% occurs in meiosis I and 23% in meiosis II. Common types of human aneuploidy include trisomy from meiosis I nondisjunction, monosomy, and uniparental disomy. In a particular type of trisomy that arises in meiosis II nondisjunction, or M2 trisomy, an extra chromosome is identical to one of the two normal chromosomes. M2 trisomy (also called mitotic trisomy) is particularly difficult to detect. There is a great need for a better method that can detect for many or all types of aneuploidy at most or all of the chromosomes efficiently and with high accuracy, especially a method that can determine aneuploidy states involving multiple identical chromosomes, such as with mitotic trisomy, or some cases of uniparental disomy.

Karyotyping, the traditional method used for the prediction of aneuploidy and mosaicism is giving way to other more high-throughput, more cost effective methods such as Flow Cytometry (FC) and FISH. Karyotyping involves the isolation of a single cell, the staining of the chromosomes in that cell, and the visualization and identification of the chromosomes. A major drawback to karyotyping is the high cost. Currently, the vast majority of prenatal diagnoses use FISH, which can determine large chromosomal aberrations and PCR/electrophoresis, and which can determine the identity of a small number of SNPs or other alleles. FISH involves the chromosome-specific hybridization of fluorescently tagged probes to cellular DNA, and subsequent visualization and quantification of the amount of fluorescent probes present. One advantage of FISH is that it is less expensive than karyotyping, but the technique is complex and expensive enough that generally only a small selection of chromosomes are tested (usually chromosomes 13, 18, 21, X, Y; also sometimes 8, 9, 15, 16, 17, 22). In addition, FISH has a low level of specificity. Roughly seventy-five percent of PGD today measures high-level chromosomal abnormalities such as aneuploidy, using FISH, with error rates on the order of 10-15%. There is a great demand for an aneuploidy screening method that has a higher throughput, lower cost, wider scope, and greater accuracy.

There are a number of methods described in the literature for determining the ploidy state (or chromosome count) of one or a number of cells that make use of genotyping. For example, Handyside (PCT filing WO/2007/0057647) discusses the concept of using measured SNP data to assemble a notional haplotype of an embryo, looking for aberrations from the expected measured SNP data given the notional haplotype, and flagging any aberrations as likely cases of aneuploidy. One drawback to these methods is that they are unable to detect ploidy states such as uniparental disomy (UPD) and mitotic trisomy, where there are two identical chromosomes in the embryo. Another drawback is that they require the haplotypes of one or both of the parents, a non-trivial issue. There exists a need for a new method that utilizes genetic information that can be gathered in a highly efficient, cost effective manner that also alleviates the inability to determine ploidy states due to multiple copies of identical chromosomes such as UPD and mitotic trisomy. Furthermore, there exists a need for a new method that utilizes genetic information that can be gathered in a highly efficient, cost effective manner that does not require the knowledge of parental haplotypes.

Using the current state of the art methods to measure genetic data may have drawbacks, such as the lack of currently known ways to amplify and measure the genetic data of one or a small number of cells, at a large number of alleles, while being able to reduce the ADO rate and/or increase the accuracy of the measurements at a subset of key alleles as compared to the entire set of measured alleles. Also, when measuring the genetic data of a diploid cell(s), it is often important to be able to determine the haplotypes of the cell. However, this may be difficult to do in an effective manner on one or a small number of cells. It may also be difficult to determine the ploidy state of a cell with high accuracy, at all of the chromosomes, and in an efficient manner with respect to costs and time.

### SUMMARY

Methods of cell genotyping are disclosed herein. In the context of whole genome amplification of small quantities of genetic material, whether through ligation-mediated PCR (LM-PCR), multiple displacement amplification (MDA), or other methods, dropouts of loci occur randomly and unavoidably. It is often desirable to amplify and genotype the whole genome nonspecifically, and at the same time increase the chance that a particular locus, or set of loci, are amplified and measured accurately. When determining the identity of a key allele (typically a disease-linked allele), there are multiple ways to increase the likelihood that the key allele is identified correctly. One way may be to use targeted primers in the amplification to raise the likelihood that the target allele is measured correctly. Another may be to use whole genome amplification techniques to measure multiple polymorphic alleles that lie within a disease gene and/or closely flanking it, enabling the deduction of the high-risk (i.e. mutation carrying) haplotypes that have been inherited by embryo. Embodiments of the present invention include methods for simultaneous loci targeting and whole genome amplification.

According to aspects illustrated herein, there is provided a method that enables one to amplify the whole genome of a single cell, or small number of cells, while biasing the amplification to amplify a set of desired loci preferentially. The addition of “spike-in” primers (locus-specific primers) may lower the likelihood that the loci of special importance are subject to ADO. In one embodiment of the invention, a method minimizes the chances that one or more single nucleotide polymorphisms (SNPs) of interest drop out during WGA using a single or small number of cells. This method may be advantageous when the number of alleles of interest may be sufficiently large that techniques such as FISH may be incapable of making allele calls at all of the alleles of interest, and array based genotyping may be necessary.

According to aspects illustrated herein, there is provided a method for determining genetic haplotypes using the genetic material from only one (1) cell, or a small number of cells, which by definition may have a known number of chromosome copies. In one aspect of this embodiment, the method avoids purifying the DNA, which may minimize DNA damage. This method may minimize DNA strand breakage, and thus lower the chance of false haplotyping. When a small number of cells are used in one reaction, this approach may further lower the risk of ADO, though it may require dividing the genetic material into a larger number of fractions. This risk may also be mitigated by using informatics based approaches, such as Parental Support™, that utilize the knowledge of genetic data measured on related individuals, and/or publicly available haplotype databases such as those supported by the Hapmap Project and from the Perlegen Human Haplotype Project, to infer genetic data not measured or measured incorrectly. The Parental Support™ method is described in U.S. application Ser. No. 11/603,406 and U.S. application Ser. No. 12/076,348 and the entirety of both of these applications are hereby incorporated herein by reference for the respective teachings therein.

According to aspects illustrated herein, there is provided a method for detecting aneuploidy by dividing the genetic matter from a single cell, or a small number of cells, into a plurality of fractions before amplification and genotyping, which may be performed for individual fractions. Since only a single copy of the genome may be present in a single cell, each fraction in which a given allele may be found implies a different homologous chromosome. From the number of fractions in which the various alleles from a given chromosome are found, it may be possible to determine the ploidy state of that chromosome in the cell. This method may allow the detection of types of aneuploidy due to multiple identical chromosomes in a cell. Additionally, this method may not require the knowledge of parental haplotypes.

The systems, methods, and techniques of embodiments of the present invention may be used to in conjunction with embryo screening in the context of IVF, or prenatal testing procedures, in the context of non-invasive prenatal diagnosis. The systems, methods, and techniques of embodiments of the present invention may be employed in methods of increasing the probability that the embryos and fetuses obtain by in vitro fertilization are successfully implanted and carried through the full gestation period, and result in healthy babies. Further, the systems, methods, and techniques of embodiments of the present invention may be employed in methods to decrease the probability that the embryos and fetuses, which are obtained by in vitro fertilization, implanted and gestated, are at risk for chromosomal, congenital or other genetic disorders.

Various embodiments provide certain advantages. Not all embodiments of the invention share the same advantages and those that do may not share them under all circumstances. Further features and advantages of the embodiments, as well as the structure of various embodiments are described in detail below with reference to the accompanying drawings.

### BRIEF DESCRIPTION OF THE DRAWINGS

The presently disclosed embodiments will be further explained with reference to the attached drawings, wherein like structures are referred to by like numerals throughout the several views. The drawings shown are not necessarily to scale, with emphasis instead generally being placed upon illustrating the principles of the presently disclosed embodiments.

FIG. 1 is a schematic illustration of an illustrative embodiment of an experimental setup for WGA using spike-in primers;

FIG. 2 is an illustration of an illustrative embodiment of a spike-in primer design;

and

FIG. 3 is a schematic illustration of an illustrative embodiment of a PCR approach.

While the above-identified drawings set forth presently disclosed embodiments, other embodiments are also contemplated, as noted in the discussion. This disclosure presents illustrative embodiments by way of representation and not limitation. Numerous other modifications and embodiments can be devised by those skilled in the art which fall within the scope and spirit of the principles of the presently disclosed embodiments.

### DETAILED DESCRIPTION

The inventions are not limited in their application to the details of construction and the arrangement of components set forth in the following description or illustrated in the drawings. The inventions are capable of being arranged in other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items. It will be readily apparent to one of skill in the art that the primers (SEQ ID NOs: 1-24) disclosed in the present application may also include the reverse complement thereof.

Aspects of the inventions are described below with reference to illustrative embodiments. It should be understood that reference to these illustrative embodiments is not made to limit aspects of the inventions in any way. Instead, illustrative embodiments are used to aid in the description and understanding of various aspects of the inventions. Therefore, the following description is intended to be illustrative, not limiting.

The headings of or in the various sections in this application are inserted for convenience only and are not intended to affect the meaning or interpretation of this application. Any disclosures in any part of this application may be employed with any disclosures in another part of the application. For example, techniques described in “Using the GR MDA Spike-In” section may be used in combination with or in lieu of techniques described in “Using the Sigma WGA Kit” section.

Some embodiments of the present invention are designed to determine the genetic data from one or a small number of cells with high accuracy. Some embodiments of the present invention include a method for whole genome amplification with targeted amplification of certain alleles of interest using “spike-in” primers. Since WGA involves generic primers, ADO rates tend to be higher than in targeted amplification, where the primers may be specifically designed for loci of interest. In some embodiments, primers are used that have been specifically designed for loci of interest in conjunction with generic primers in the context of WGA, such that it may be possible to realize the benefit of both methods: amplification of the entire genome as well as high accuracy amplification of loci of interest.

Some embodiments may include a method for determining the haplotype of a single or small number of cells by division of the DNA before amplification. In some aspects of this invention, the free genetic material (e.g., blastomeric, etc.) from one or a small number of cells may be divided into a sufficiently large number of fractions such that it may be unlikely to find more than one haplotype per sample. Since the majority of the haplotypes may be in different fractions, and each fraction may be amplified and genotyped individually, so that the individual haplotypes may be measured separately using standard genotyping methodology. In some embodiments, techniques may be used to minimize the DNA strand breakage, thus increasing the chance that large unbroken haplotypic sections may be measured in individual wells and enhancing the ability to reconstruct the entire haplotype of the target from the genotypes measured from the individual fractions.

Some embodiments may include a method for determining the ploidy state of a cell, at some or all of the chromosomes, by dividing the genetic material from a single cell into a plurality of fractions, and then separately amplifying and genotyping each fraction. By determining the number of fractions in which a given allele may be detected, for many alleles on a given chromosome, it may be possible to determine the number of chromosomes present in the cell. The distribution of the number of fractions in which each allele is found, for a set of alleles on a given chromosome, may be indicative of the ploidy state of a given chromosome. In some aspects, the ploidy state can be determined either by observing the maximum number of fractions that alleles from a given chromosome are found, and taking that to be the ploidy state. In some aspects, the ploidy state may be determined by comparing the observed distribution of alleles to the expected distributions for different ploidy states, given the conditions of the experiment, and taking the actual ploidy state to be the one whose expected distribution most closely matches the observed distribution.

The present methods may be relevant in the context of Gene Security Network's proprietary Parental Support™ (PS) method. The Parental Support™ method uses the measured genetic data from a single or small number of cells, along with parental genetic data and the knowledge of a mechanism of meiosis, as inputs to determine the genotype at a plurality of alleles, and the ploidy state of an embryo, of a fetus, or of any target cell or group of cells. The spike-in method described herein may generate data that may be optimized in the context of being used as input for the Parental Support™ method. The haplotyping method described herein may determine haplotypes that can be used in the context of determining parental or target haplotypes for use as input for the Parental Support™ method. The method for determining ploidy states may be used to augment or replace aspects of the Parental Support™ method.

In some embodiments, the subject, such as a target individual, may be an embryo, and the purpose of applying the disclosed method to the genetic data of the embryo may be to allow a doctor or other agent to make an informed choice of which embryo(s) should be implanted during IVF. In some embodiments, the target individual may be a fetus, and the purpose of applying the disclosed method to genetic data of the fetus may be to allow a doctor or other agent to make an informed choice about possible clinical decisions or other actions to be taken with respect to the fetus. In some embodiments, the target individual may be a fetus, and the nucleated fetal cell(s) may be isolated in a non-invasive manner from maternal blood.

### Definitions

Genotyping: may be the genetic constitution of a cell or a subject (i.e. the specific allele makeup of the subject), usually with reference to a specific character under consideration. The term may also refer to a subject's specific genomic sequence, or a representative genomic sequence of a species or group; a genotype may be a measurement of how an individual differs or may be specialized within a group of individuals or a species. A subject's genotype can be measured with regard to a particular gene or genes of interest, including the location, such as the number chromosome on which the gene may be located, which of the two homologous chromosomes the gene may be located on, and where along the chromosome the gene may be located, and the identity, such as the sequence of base pairs that may make up the gene. The genotype may also refer to the number and origin of chromosomes in the subject's genome. The term genotype may also include non-hereditary DNA mutations that are not classically understood as representing a subject's genotype. For example, the term may be meant to apply to the genotype of a particular cancer, wherein the genotype of the disease may be distinct from the genotype of the subject that has the cancer.

SNP (Single Nucleotide Polymorphism): may be a single nucleotide that may differ between the genomes of two members of the same species. As used herein, there may not be any limit on the frequency with which each variant occurs.

Locus: also referred to as an “allele”; may be a particular region of interest on the DNA of an individual, which may refer to a SNP, the site of a possible insertion or deletion, or the site of some other relevant genetic variation. Disease-linked SNPs may also refer to disease-linked loci or disease-linked alleles.

To call an allele: may mean to determine the state of a particular locus of DNA. This may involve calling a SNP, determining whether an insertion or deletion is present at that locus, determining the number of insertions that may be present at that locus, or determining whether some other genetic variant is present at that locus.

To clean genetic data: may mean to take imperfect genetic data and correct some or all of the errors or fill in missing data at one or more loci.

Segment of a Chromosome: may be a section of a chromosome that can range in size from one base pair to the entire chromosome; also called a “minichromosome.” Chromosome: may refer to either a full chromosome, or also a segment of a chromosome.

Haplotypic Data: also called “phased data” or “ordered genetic data”; may be data from a single chromosome in a diploid or polyploid genome, such as the segregated maternal or paternal copy of a chromosome in a diploid genome.

Unordered Genetic Data: may be pooled genetic data derived from measurements on two or more chromosomes in a diploid or polyploid genome, such as both the maternal and paternal copies of a chromosome in a diploid genome.

Genetic data ‘in’, ‘of’, ‘at’ or ‘on’ an individual: may mean the data describing aspects of the genome of an individual and may refer to one or a set of loci, partial or entire sequences, partial or entire chromosomes, or the entire genome.

Target Individual: also called “subject”; may be a subject or individual whose genetic data is being determined or otherwise analyzed. “Target individual” may refer to an adult, a juvenile, a fetus, an embryo, a blastocyst, a blastomere, a cell or set of cells from an individual, from a cell line, or any set of genetic material. The target individual may be alive, dead, frozen, or in stasis. In some embodiments, the genetic data may be from humans, while in some embodiments, the target individual may be any other DNA containing organism. In some embodiments, the target individual may be non-human vertebrates (e.g., dogs, cats, horses, cows, pigs, etc.), companion animals (e.g., dogs, cats, hamsters, etc.), livestock (cows, horses, sheep, etc.), the production of “cultivated” animals (e.g. race horses, “pure-bred” varieties of dogs or cats, etc.), or any other nucleic acid containing organism.

Related Individual: may be any individual who is genetically related, and thus may share haplotype blocks with the target individual. Some examples of related individuals include biological father, biological mother, son, daughter, brother, sister, half-brother, half-sister, grandfather, grandmother, uncle, aunt, nephew, niece, grandson, granddaughter, cousin, clone, the target individual himself/herself/itself, and/or other individuals with known genetic relationship to the target. The term “related individual” may also encompasses any embryo, zygote, fetus, sperm, egg, blastomere, blastocyst, or polar body derived from a related individual.

Whole Genome Amplification (WGA): may be a technique designed to amplify all or some of the DNA in a sample. Some methods to perform WGA may include the commercially available GE MDA kit and the Sigma WGA kit. Some other methods may include degenerated oligonucleotide primed PCR (DOP-PCR) and ligation mediated PCR (LM-PCR).

WGA kit (Sigma): may be a commercially available kit for conducting whole genome amplification.

Free DNA: may mean DNA which is not contained by a cell wall.

Allele Drop Out (ADO): may mean a situation that may occur during genotyping where an allele fails to amplify and the expected allele may not be measured correctly. An ADO may be a false negative when measuring genotypic data. In the case of a homozygous allele, it may not be possible to recognize an ADO that is not also a LDO; in the case of a heterozygous allele that is not known to be heterozygous, it may not be possible to differentiate between an ADO and the case that that allele is homozygous.

Allele Drop In (ADI): may mean a situation that may occur during genotyping where an allele is measured to have a certain identity, but where that measurement may be incorrect and the actual genetic material may support that determination. An ADI may be a false positive when measuring genotypic data.

Locus Drop Out (LDO): may mean a situation that may occur during genotyping where both of two homologous alleles fail to amplify.

Spike-in: may mean inclusion of locus-specific oligonucleotide primers to the usual reagents used during whole genome amplification.

Phasing: may mean an act of determining the haplotypic genetic data of a target individual given unordered, diploid genetic data, or other genetic data.

One or a small number of cells: may mean one cell, two cells, up to five cells, as many as twenty cells, more than twenty cells, any number or range in between, or any combination thereof, as not all embodiments are intended to be limited in this manner. Note this also may refer to an amount of free DNA (such as in the case of non-invasive prenatal diagnosis) that may approximately correspond to the amount of DNA found in one or a small number of cells.

Ploidy calling: also chromosome copy number calling, may be an act of determining the number and identity of chromosomes present in a cell.

Ploidy State: may be the number and identity of one or more chromosomes in a cell.

Base Pair (bp): may be an elementary unit of DNA; 1 kb equals 1,000 base pairs; 1 Mb equals 1,000,000 base pairs.

Nucleic Acid: may be any macromolecule composed of chains of monomeric nucleotides and may carry genetic information or form structures within cells. Examples of nucleic acids include deoxyribonucleic acid (DNA) and ribonucleic acid (RNA). Nucleic acids may also include artificial nucleic acids, such as peptide nucleic acid (PNA), Morpholino and locked nucleic acid (LNA), as well as glycol nucleic acid (GNA) and threose nucleic acid (TNA). Nucleic acids may also include any nucleobases, nucleosides, nucleotides and deoxynucleotides.

Some embodiments of the invention can be used in conjunction with the informatics-based approaches, such as Parental Support™. The Parental Support™ method may be used to determine the genetic data, with high accuracy, of one or a small number of cells, specifically to determine disease-related alleles, other alleles of interest, and/or the ploidy state of the cell(s).

The Parental Support™ technology may use genetic measurements of the parents to improve the reliability of allele calls and ploidy calls on the child cells. To improve the accuracy in calling an allele derived from the mother, in the absence of maternal haplotype information, the Parental Support™ may use non-phase data of multiple blastomeres (preferably three or more) in order to determine which segments of which maternal chromosomes contributed to the embryos and to indirectly phase the genetic data of the mother. Paternal haplotype data can be obtained by measuring sperm cells, as they each contain only one copy of each chromosome. Maternal haplotypes may be more difficult to determine. Embodiments of the invention described herein may enable direct measurement of the maternal haplotype. In this way, the measurements of multiple different children or embryos may not be necessary to infer the maternal haplotype and the ploidy status and allele calls from a sample of a single child cell, such as a single blastomere or a single fetal sample, may be reliably determined.

The Parental Support™ method may enable the cleaning of incomplete or noisy genetic data using the genetic data of one or more related individuals as a source of information. It may also enable the determination of chromosome copy number using said genetic data. The Parental Support™ method may be particularly useful in the context of facilitating diagnoses focusing on inheritable diseases, chromosome copy number predictions, increased likelihoods of defects or abnormalities, as well as making predictions of susceptibility to various disease-and non-disease phenotypes for individuals to enhance clinical and lifestyle decisions.

The Parental Support™ method may make the use of known parental genetic data, such as haplotypic and/or diploid genetic data of the mother and/or the father, together with the knowledge of the mechanism of meiosis and the imperfect measurement of the target DNA, in order to reconstruct, in silico, the target DNA at the location of key loci with a high degree of confidence. The Parental Support™ method can reconstruct not only SNPs that were measured poorly, but also insertions and deletions, and SNPs or whole regions of DNA that were not measured at all. Furthermore, the Parental Support™ method can both measure multiple disease-linked loci as well as screen for aneuploidy, from a single cell.

The haplotypic genetic data and ploidy data that can be generated by the methods of measuring the amplified DNA from one cell using the methods described herein can be used for multiple purposes. For example, in the context of preimplantation genetic diagnosis or prenatal diagnosis, they can be used for detecting aneuploidy, uniparental disomy, sexing the individual, as well as for making a plurality of phenotypic predictions based on phenotype-associated alleles. Currently, in IVF laboratories, due to the techniques used, it may be the case that one blastomere can only provide enough genetic material to test for one disorder, such as aneuploidy, or a particular monogenic disease. Since some embodiments of the methods disclosed herein and Parental Support™ may have the common step of measuring a large set of SNPs from a single or small number of cells, regardless of the type of prediction to be made, a physician, parent, or other agent may not be limited to a single or small number of disorders for which to screen. Instead, the option may exist to screen for as many genes and/or phenotypes as the state of medical knowledge will allow. With the disclosed method, one advantage to identifying particular conditions to screen for prior to genotyping the blastomere is that if it is decided that certain loci are especially relevant, then a more appropriate set of SNPs which are more likely to co-segregate with the locus of interest can be selected. Additionally, one or a set of targeted locus-specific primers can be included, as described elsewhere in this disclosure. Both of these actions can increase the likelihood that alleles of interest will be measured accurately.

### WGA Spike-In

In some embodiments, a method of performing whole genome amplification on a DNA sample from a target individual may include: adding one or more spike-in primers to the DNA that target one or more loci of interest; and amplifying the DNA using a method for whole genome amplification; wherein the addition of the spike-in primers decreases the likelihood of allele drop out at the one or more loci of interest.

In one aspect of this embodiment, the DNA sample may be a single cell, two cells, 3-5 cells, more than five cells, or fetal DNA isolated from maternal blood. In an aspect, the DNA sample may be from more than 10, more than 20, more than 50 cells. In another aspect of this embodiment, the amplification may be done using a WGA kit from Sigma or a GE MDA kit. In an aspect, the amplification may include using a commercial whole genome amplification kit. In an aspect, the whole genome amplification kit can be a non-commercial preparation, or a combination of commercially-available and non-commercially available reagents for whole genome amplification.

In another aspect of this embodiment, there may be a single locus of interest, 2-5 loci of interest, 5-10 loci of interest, 10-15 loci of interest, 15-20 loci of interest, or more than 20 loci of interest. In another aspect of this embodiment, the spike-in primer may be designed to amplify a product between about 200 bp and about 1000 bp, or about 600 bp. In another aspect, the spike-in primer may be designed to amplify a product of about 300 bp, of about 400 bp, of about 500, of about 700 bp, of about 800 bp, of about 900 bp, of about 1000 bp, of about 1100 bp. In another aspect of this embodiment, the likelihood of allele drop out may be decreased by up to about 20%, up to about 25%, up to about 30%, up to about 35%, up to about 40%, up to about 45%, up to about 50%, up to about 55%, up to about 60%, up to about 65%, up to about 70%, up to about 75%, up to about 80%, up to about 85%, up to about 90%, up to about 95%, or over 95%.

In another aspect of this embodiment, the method further includes synthesizing the spike-in primers. In another aspect of this embodiment, the method further includes measuring the genotype of the amplified DNA. In another aspect of this embodiment, the method is used in combination with an informatics method such as the Parental Support™ method.

When DNA from a single or small number of cells may be amplified in the context of whole genome amplification, for example using the WGA kit from Sigma or the GE MDA kit, a problem may be allele drop out (ADO). ADO rates may be between 20% and 60%, and can range anywhere from more than 0% to 100%. The reasons for ADOs may not be entirely known, ADOs can occur both in a random manner (unpredictable) and systematically (e.g., high GC containing sequences are more difficult to amplify and may drop out more frequently). Events in both lysis and amplification may contribute to ADO, such that chromatin may not be released from histones during lysis, part of genome may be trapped on equipment, or the amplification if biased against certain regions. In one aspect of this embodiment, even when ADO occurs at a locus of interest, the locus can be inferred by calling the alleles on either side of the locus of interest. In some aspects of the invention, the spike-in primers may be designed to amplify not only the locus of interest, but also a number of alleles on either side of said locus. In some aspects of the invention, the spike-in primer may be designed to amplify about 100 bp to about 500 bp on either side of each locus of interest. In some aspects of the invention, the spike-in primer may be designed to amplify about 250 bp to about 300 bp on either side of each locus of interest. In some aspects of the invention, the spike-in primer may be designed to amplify about 300 bp on either side of each locus of interest. In some aspects of the invention, the spike-in primer may be designed to amplify from about 50 bp, about 100 bp, about 150 bp, about 200 bp, about 250 bp, about 300 bp, about 350 bp, about 400 bp, about 450 bp, about 500 bp, about 550 bp or more than about 550 bp on either side of each locus of interest.

The addition of “spike-in” primers during WGA may significantly lower the likelihood that the loci of interest are subject to ADO. In some embodiments of the present invention, primers targeting regions surrounding the SNPs of interest may be included in the WGA assay. In some embodiments, the WGA may be done using a WGA kit from Sigma. In some embodiments, the WGA may be done using the GE MDA kit. In some embodiments of the invention, the WGA can be done using, but not be limited to: preamplification of particular loci before generalized amplification by MDA or LM-PCR, the addition of targeted PCR primers to universal primers in the generalized PCR step of LM-PCR, and the addition of targeted PCR primers to degenerate primers in MDA. In some embodiments, the MDA-type kit may be REPLI-g Kit (Qiagen). In some embodiments, the WGA assay may be done using PEP-PCR (primer extension pre-amplification PCR) or DOP-PCR (degenerate oligonucleotide primer PCR). In some embodiments, the targeted primers may be added at the same time as the generic primers, or before the generic primers so that they can preferentially hybridize to the target regions of DNA.

Different WGA methods may require different timing of primer introduction. In some embodiments of the invention, the Sigma WGA kit may be used and the spike-in primers may be added in the amplification. In one experiment using an embodiment, spike-in of a single primer pair reduced LDO rates from 79% to 7%. In a multiplex spike-in, the average LDO decreased from 29% to 9%. In another embodiment of the invention, the GE MDA kit may be used, and the spike-in primers may be added in the lysis. In one experiment using an embodiment, spike-in of specific primers to the lysis reaction reduced LDO rates from 26% to 7% for a single spiked-in primer, and to 8% for multiplex spiked-in primers. Data relating to the above-described embodiments may be found in Tables 2, 3 and 4.

Some embodiments of the present invention may involve measuring the polymorphic alleles flanking the alleles of interest and using the identity of the flanking alleles to determine which parental haplotype is present in a cell. Using this method, identity of any incorrectly measured alleles of interest can be inferred. In some embodiments, the alleles of interest may be disease-linked alleles. While these embodiments can mitigate the problems associated with incorrectly measured alleles of interest, it may still be desirable to maximize the accuracy with which the desired disease-linked, or otherwise targeted alleles may be measured. In some embodiments of the invention, the method of measuring the one or more alleles of interest with maximal accuracy may be combined with the method of measuring the flanking polymorphic alleles to make accurate genetic determinations.

In some embodiments of the invention, the method for determining the genetic data from the whole genome, while biasing the amplification to reduce the ADO rate at certain alleles of interest, may be used in conjunction with an informatics based approach, such as the Parental Support™ method. This combination can be used to determine the identity of certain disease-related alleles or other alleles of interest, and/or to determine the ploidy state of the individual, with maximal accuracy. In some embodiments of the invention, the determination of the identity of the alleles of interested and/or ploidy state may be done for the purpose of choosing which embryo(s) to implant or not implant, or to making clinical decisions regarding a fetus. The higher the accuracy of the genetic data, the more accurate the predictions of the informatics based approach may be able to make. Embodiments of the invention may provide optimized genetic data for the informatics based method, such that the accuracy of the subsequent predictions may be maximized.

### Using the Sigma WGA Kit

In some embodiments of the invention, the WGA may be performed using a WGA kit from Sigma in combination with specific spike-in primers. Performing WGA using the Sigma WGA kit may entail the following general steps: (1) cell lysis and DNA preparation, (2) addition of Library Preparation mix, and (3) amplification (includes addition of Amplification mix). The DNA preparation may involve neutralization or purification, and the amplification step may be repeated. In one aspect of this embodiment, the spike-in primers may be included in the “amplification” step alongside the regular WGA primers. The inclusion of the spike-in primers into the “Library Preparation” step (most likely where adaptors are attached to DNA fragments) may have no effect on the target's ADO rate, possibly because they too were tagged by adaptors (they were included here initially due to this steps' temperature profile).

In some embodiments of the invention, the primers may be added into the PCR amplification mix. In these embodiments, the inclusion of locus specific oligonucleotides (primers) may result in the bias of amplification towards the regions of interest. The effect may be lower ADO rates of targeted loci compared to ADO rates of the untargeted loci. In one aspect of this embodiment, the primers may be located so that the PCR product can be amplified along with the method's PCR—which may span 200-1500 bp. In one aspect of this invention, the spike-in primer pairs may be between about 200 bp and about 1500 bp. In another aspect of this invention, the spike-in primer pairs may be from about 400 bp to about 1200 bp. In another aspect of this invention, the spike-in primer pairs may be from about 500 bp to about 1000 bp. In another aspect of this invention, the spike-in primer pairs may be from about 600 bp to about 750 bp. In another aspect of this invention, the spike-in primer pairs may be about 600 bp.

The efficiency of the amplification was evaluated by performing a 2^{nd} targeted PCR with only the spiked-in primers, where the DNA produced in the initial amplification was used as the source DNA for the second PCR. Thus, if the targeted alleles amplified as intended during the initial WGA with spiked-in primers, then the second PCR may be expected to produce a product corresponding to the size of the designed initial primer. A combination of TAQMAN, which can determine the exact identity of a given SNP, and information obtained from agarose gels, which can detect LDO, was used to differentiate between a correctly called homozygous allele and a heterozygous allele measured with ADO.

In one aspect of this invention, primers may be designed and synthesized for each locus of interest. In this aspect, the primers may be designed towards regions of approximately 300 bp on each side of the SNP, or towards regions immediately upstream and downstream, of their SNP to the sense (+) and non-sense (−) genomic strands, respectively (See Table 1). In one aspect of the invention, the concentration of the spike-in primers may be about equal to the primer concentration in regular PCR. In one aspect of this embodiment, the concentration of the spike-in primers may be approximately 250 nM. In another aspect of this embodiment, the concentration of the spike-in primers may be between about 100 nM and about 350 nM. In some aspects of the invention, the concentration of spike-in primers may be about 10 nM, about 25 nM, about 50 nM, about 100 nM, about 150 nM, about 200 nM, about 250 nM, about 300 nM, about 350 nM, about 400 nM, less than 10 nM, more than 400 nM or any range therebetween.

TABLE 1
PCR primers used in the spike-in experiments (5′-3′).
Numbers represent loci using ABI TAQMAN probe annotations.
Primer sequences, 600 by spike-ins

| | | | |
|---|---|---|---|
| 656642 Fw | ttctctgtgcctctgttttcttg | 656763 Fw | aggaatttggttgtggagagc |
| SEQ ID NO: 1 | | SEQ ID NO: 7 | |
| 656642 Rev | tagctcatgtccctgcctctc | 656763 Rev | acctgtcttccctcccagtt |
| SEQ ID NO: 2 | | SEQ ID NO: 8 | |
| 2540897 Fw | tacttgtcctgctgcgatgt | 13096 Fw | atgtggccacttgaaaaagaa |
| SEQ ID NO: 3 | | SEQ ID NO: 9 | |
| 2540897 Rev | ttccccaggacattaagagcg | 13096 Rev | acccacccctagaggttctga |
| SEQ ID NO: 4 | | SEQ ID NO: 10 | |
| 2668640 Fw | tttgtttgttttgctgcttgc | 2602208 Fw | tgttccaggcactgaacaaca |
| SEQ ID NO: 5 | | SEQ ID NO: 11 | |
| 2668640 Rev | cctctccctccaactccaatt | 2602208 Rev | aacgctgtgccagattctctt |
| SEQ ID NO: 6 | | SEQ ID NO: 12 | |

### Using the GE MDA Spike-In

As described above, the idea of including (spike-in) locus specific oligonucleotides (primers) may be to bias amplification towards the regions of interest, with the effect of lowering ADO rates of the targeted loci compared to non-spiked amplifications, while leaving non-targeted loci unaltered. In one embodiment of the invention, the WGA may be conducted using a GE MDA kit and spike in primers. Performing WGA using the GE MDA kit may entail the following general steps: (1) cell lysis and DNA preparation, (2) amplification (includes addition of MDA mix), and (3) heat inactivation of enzyme. The DNA preparation may involve neutralization, purification, putting the DNA into sample buffer, and/or denaturation.

Not all embodiments of the multiple displacement amplification may be limited in this manner. In some embodiments, the step of multiple displacement amplification may include using a commercial multiple displacement amplification kit. In some embodiments, the multiple displacement kit can be a non-commercial preparation, or a combination of commercially-available and non-commercially available reagents for multiple displacement amplification.

In some embodiments, the targeted primers may be added during the cell lysis step. In one aspect of this embodiment, the targeted primers may be added to the lysis solution. Addition of spike-in primers during MDA may serve to decrease the ADO rate at alleles of interest. Inclusion of the targeted primers into the MDA kit may have no effect. In one aspect of this embodiment, the lysis buffer solution may not be alkaline. If the lysis solution is an alkali solution, in another aspect of this embodiment, a neutralization buffer may be added prior to MDA mix to allow primer hybridization along with some time for proper hybridization. Either the alkali buffer or the neutralization solution may contain the spike-in primers. In another aspect of this embodiment, the composition of the lysis buffer may be unknown. In this aspect, the method may involve the addition of Tris-HCl, KCl and/or MgCl₂ to the lysis solution. Both the neutralization of the lysis buffer and the addition of Tris-HCl, KCl and MgCl₂ were found to increase scoring rates of the targeted SNPs. In another embodiment of the invention, dithiothreitol (DTT) may be added to the lysis step.

In another embodiment of this invention, primers may also be designed to target between 1 kb and 5 kb on each side of the target SNP. In another embodiment of this invention, primers may be designed to target between 2 kb and 3 kb on each side of the target SNP. In another embodiment of this invention, primers may be designed to targets approximately 2.5 kb on each side of the target SNP. In another embodiment of this invention, primers of a length between 300 bp and 900 bp may be added into the GE MDA kit during the lysis step. In another embodiment of this invention, primers of a length between 500 bp and 700 bp may be added into the GE MDA kit during the lysis step. In another embodiment of this invention, primers of approximately 600 bp length may be added into the GE MDA kit during the lysis step.

Thus, the primers may need to be targeted to regions at proper distances from the SNPs. A spike-in primer pair (forward and reverse, i.e. towards sense and non-sense strand, which was superior to using only one primer per locus) designed to amplify a product of approximately 600 bp may work better than a primer pair designed for a 70 bp product, or 5 kb product. This can most likely be explained by how the MDA is functioning; MDA requires certain lengths of DNA strands to amplify efficiently, but strands of too great a length may suffer breaks or polymerase fall-off. In one embodiment of the present invention, the spike-in primer pair may be designed to amplify a product of between about 50 bp and about 1000bp. In another aspect, the primer pair may be designed to amplify a product between about 100 bp and about 750 bp. In another aspect, the spike-in primer pair may be designed to amplify a product between about 200 bp and 600 bp. In another aspect, the spike-in primer pair may be designed to amplify a product about 500 bp. In another aspect, the spike-in primer pair may be designed to amplify a product about 600 bp. In another aspect, the spike-in primer pair may be designed to amplify a product about 150 bp, about 200 bp, about 250 bp, about 300 bp, about 350 bp, about 400 bp, about 450 bp, about 500 bp, about 550 bp, about 600 bp, about 650 bp, about 700 bp, about 750 bp, about 800 bp, less than about 150 bp or more than about 800 bp, as not all embodiments of the present invention are intended to be limited in this manner. In one embodiment, primers may be designed towards regions approximately 300 bp upstream and 300 bp downstream of their SNP to the sense (+) and non-sense (−) genomic strands, respectively (see Table 1).

### Single Cell Haplotyping by Division

A challenge to preimplantation genetic diagnosis may be to determine the haplotype of the embryo from the non-phased genetic data measured from the single cell blastomere. Paternal haplotype determination may be relatively straightforward to obtain by measuring sperm cells, as they each contain only one haplotype. However, maternal haplotypes may be more difficult to determine. In this context, there may be a great need to determine haplotypes in an efficient manner. Some embodiments of the present invention provide solutions to these issues.

Some embodiments of the invention include a method for determining the haplotype of a DNA sample, which may include: dividing the DNA into a plurality of fractions; genotyping the DNA in each fraction individually; and reconstructing the haplotype of the one or more cells using the genotypes determined in the genotyping step.

In one aspect of this embodiment, the DNA sample may contain DNA from a single cell, from two cells, or from three to five cells. In another aspect of this embodiment, the DNA may be divided into from two to five fractions, from six to ten fractions, from eleven to twenty fractions, from twenty-one to fifty fractions, or over fifty fractions. In another aspect of this invention, the DNA may not be purified before being divided in the dividing step. In another aspect of this invention, the DNA may be handled with minimal or no mechanical agitation. In another aspect of this invention, the DNA may originate from one or more recently lysed cells.

In another aspect of this invention, the method may further include the step of amplifying the DNA in each of the fractions before it is genotyped in the genotyping step. In another aspect of this invention, the method may further include the step of diluting the DNA before dividing it into fractions in the dividing step. In one aspect of this embodiment, the DNA may be diluted down to from about 0.01 to about 0.2, from about 0.2 to about 0.4, or from about 0.4 to about 0.8 copies of the genome per well. In another aspect of this embodiment, the DNA may be diluted down to about equal to or less than one chromosome per well. In some embodiments, the DNA may be diluted down to less than about 0.01 copies of the genome per well. In some embodiments, the DNA may be diluted to more than about 0.8 copies of the genome per well.

Some embodiments of the invention may involve a method that allows the haplotype of a single or small number of cells to be determined. Typically, using prior art methodology, when a single or a small number of cells are amplified, efforts are made to keep the DNA concentrated so as not to lose any information. In contrast, in some embodiments, the methods may take the same minimal amount of genetic material, dilute it and then divide it into a plurality of individual fractions, which then are each amplified and genotyped. In some embodiments, the genetic material may be diluted down to approximately 0.1 copy of the genome per reaction well, followed by amplifying the DNA present in the sample(s). In some embodiments, individual copies of alleles may be isolated in separate wells of a multi well plate. In some embodiments of the invention, the DNA may be divided into between five and twenty wells. In another aspect, the measured genetic data from a plurality of DNA division, amplification, and genotyping assays may be combined to give overlapping haplotype data so that the entire haplotype of the target can be reconstructed.

In some embodiments, a bioinformatics approach, such as the Parental Support™ method, may be used after amplification and genotyping to reassemble the haplotype as needed. In some embodiments, the starting genetic material may be from one cell. In another embodiment, the genetic material may be from five or fewer cells. In some embodiments of the invention, a plurality of single cells may be run in parallel, and the haplotypic information obtained from the parallel genotyping steps may be combined. In some embodiments, the genetic material may be from a blastomere. In some embodiments of the invention, the genetic material may be from a fetal cell.

In some embodiments, the method may be used to link genetic markers to a disease. In some embodiments, only one cell may be available for analysis. In another aspect, one to five cells may be available for analysis. In another aspect, the analysis may not be limited by cell number constraints. In some embodiments, the method may be used to analyze individual cells from a tumor or other tissue biopsy wherein the tissue sample may be heterogeneous. Genotyping heterogeneous samples using methods described in the prior art may involve using multiple cells as the source of genetic material. In the case of genetically heterogeneous tissue, this would give ambiguous and/or inaccurate data, as cells with different genotypes could not be differentiated. In some embodiments, the methods disclosed herein may allow the determination of the various haplotypes present in the heterogeneous sample. In this aspect, more than one haplotype may be determined from a single tissue sample that contains at least two cells.

Dividing the cellular DNA into multiple fractions before amplification and genotyping may be a method designed to mechanically separate the haplotypes so that they can be read separately. This method may be used when one or a small number of cells are used, or a very dilute solution of DNA may be used. For a euploid cell, assuming no ADO or ADI, the chance that the analogous haplotypes will be found in a separate fraction is (N-1)/N, wherein N is the number of fractions. For example, when N=3, this is ⅔, for N=10, this is 9/10.

In some embodiments of the invention, for PCR-based haplotyping, the labor intensive emulsion phase PCR may be replaced with a simple dilution process to ensure that single DNA strands are analyzed in each reaction. This embodiment may avoid the need for advanced bioinformatics, for example as described in Konfortov, et al. In one embodiment of the invention, the genetic information may be measured using genotyping arrays. In another embodiment of the invention, the bioinformatics process may be replaced with simple Sanger sequencing by using the PCR approach. In another embodiment of the invention, the method may include a modification to the Wetmur et al. primer design that avoids the reliance on forming a fused PCR product using genomic sequence located next to the primer sites. Instead, in this embodiment, the process of designing primers may be simplified by replacing the “shared” sequence with a degenerate sequence (not present in the human genome) so that it may easily be used in any subsequent primer designs.

The first step in preparing the targeted primer may be to obtain genomic DNA containing the target allele. This may be typically done by taking parental cells, lysing the cells, and amplifying them. The average DNA fragment size is approximately 20 kb in each fraction. Then, the sequence surrounding two or more SNPs of interest may be amplified. In the amplification, there may be tails of random DNA sequence on some of the primers. The random DNA sequence on the amplified sequence surrounding one SNP of interest may be complementary to the random DNA sequence surrounding the other SNP such that during PCR the two separate amplicons anneal to each other and then form a larger “minichromosome”. Because the DNA is diluted, it may be unlikely that more than one haplotype may be in the same tube. Thus, there may be effectively a single molecule of DNA in the tube and the mini-chromosome intended for amplification corresponds to a haplotype on a single DNA molecule. In one aspect of the invention, the DNA may be divided into a plurality of fractions and those fractions may be contained in wells in a microtiter plate. In another aspect of the invention, the fractions may be contained in plastic or glass tubes.

In one aspect of this embodiment, the DNA may be handled as little as possible after cell lysis. In one aspect, the DNA may not be purified, it may not be vortexed, it may not be shaken, it may not be processed, and/or it may not be mechanically mixed. A benefit of this avoidance of agitation may be that the chromosomal breakage may be minimized.

In one aspect of this invention, before splitting the sample containing a single genome into individual fractions, the sample may be essentially homogeneous, i.e. all chromosome copies may be essentially equally distributed in the dilution. In one aspect of this embodiment, the cell may be initially placed in lysis buffer solution (typically 5 μl), after which, the MDA mix may be added, which also serves as dilution buffer, and the force of addition of the MDA mix may be sufficient to mix the solution. In one aspect of this embodiment, the MDA mix may be added gently—with as little disturbance to the lysis solution as possible. In another aspect of this embodiment, the combination of the MDA mix and the lysis solution may be done manually—by slowly and gently agitating the solution. In this aspect, as opposed to standard protocol, the mixing of the MDA mix and the lysis solution may not be done with a vortex. The use of a vortex, even for one second, may have a negative effect on DNA integrity.

In another aspect of this embodiment, the reaction may be kept at room temperature or on ice from about one minute to about five minutes, prior to being placed at 30° C. Without being bound by a particular mechanism, it may be thought that the longer pre-incubation time may have a beneficial effect on how the genome is homogenized by simple Brownian motion. In another aspect of this invention, the MDA mix may be supplemented with PBS to minimize depurination and beta-elimination that may cause DNA breakage (Molina et al., Biochem. Biophys. Acta, 2007, 1768(3), 669-677). In one embodiment of the invention, the after mixing the MDA mix and the lysis buffer, the reaction may be left at room temperature or on ice for about five minutes. In another embodiment, the reaction may be left at room temperature or on ice for at least one minute. In one embodiment of the invention, the DNA source may be from cell culture.

In some embodiments, the DNA may not be diluted prior to partitioning into multiple fractions. In one aspect, adequate separation may be ensured by directly enforcing a limit on the volume or weight of genetic material present, and/or indirectly by setting a limit on the osmotic pressure. In some embodiments, a mini-chromosome may not be formed during the haplotyping, instead the segments may be measured on a SNP by SNP basis, and the target haplotype may be reconstructed statistically.

In some embodiments of the invention, single cells (from whole blood, tissue culture, or other sources) may be sorted through several wash droplets and placed in lysis buffer. In one embodiment more than one cell may be processed to alleviate the risk of failure in single cell sorting (e.g. the cell sticks to pipette, etc). In one aspect of this embodiment, the lysis buffer may contain Proteinase K and/or DTT, thereby encouraging homogeneous distribution in the subsequent dilution. In one embodiment, a Proteinase K may be used that is mostly or completely inactivated at a relatively low temperature for a short time.

In an aspect of this embodiment, pipetting out from the lysis reaction may be minimized, as that can result in loss of genetic material. In one aspect, since MDA (GE) is composed of mainly Sample Buffer and Reaction Buffer and is adversely effected if diluted, the MDA reaction mix may be used as dilution buffer of the lysed cell. In one embodiment of the invention, to reduce breakage of genetic material, the whole reaction mix may also contains 1× PBS. In one embodiment, the addition of the MDA mix may be made with an intermediate force, i.e. not too fast (so that lysis and MDA is mixed vigorously) and not too slow (so that no mixing takes place), at a point above the lysis volume not to make contact with the pipette tip. In this aspect, the reaction mix may be typically left at room temperature for several minutes. In one embodiment of the invention, the sample may be left to sit for a period of time between thirty seconds and thirty minutes. In another embodiment the sample may be left to sit for a period of time between one and ten minutes. In one embodiment, the sample may be left to sit for a period of time between two and four minutes. In one embodiment the sample may be left to sit on ice while the mixing takes place. Using the same pipette tip to avoid loss of genetic material, the reaction mix may then be split into equal volumes. For example, a split into five portions may give a 20% chance (⅕) of receiving both copies of a chromosome in the same portion. Each portion may be treated as a separate MDA reaction and analyzed separately, for example using TAQMAN, PCR, real-time PCR with SYBR detection, or ILLUMINA arrays, or by other means. In the case where other whole genome amplification methods are used, the same approach may be taken if possible: the lysis may be diluted with the amplification reagents and then split, in order to reduce the risk of losing genetic material due to pipetting.

In some embodiments of the invention, in order to increase the possibility of complete coverage of all chromosomes, this method may be used on a plurality of cells in parallel. In this aspect, the information from different reactions may be used if stretches of separate but neighboring haplotype blocks with overlapping sections can be found. In one embodiment of the invention, two to twenty cells may be run in parallel; in another embodiment, four to eight cells may be run in parallel. In another embodiment the plurality of cells may be processed in the same reaction volume and the number of fractions into which the cellular DNA may be divided is adjusted accordingly.

### Ploidy Calling

Some embodiments of the invention may include a method for determining the ploidy state of one or more chromosomes of a single cell containing nuclear DNA, wherein a given chromosome is associated with a known set of alleles. The method may include: dividing the DNA into a plurality of fractions; genotyping the DNA in each of the fractions; determining the number of fractions in which each allele that is associated with a given chromosome is detected; and using the data from the determining step to determine the ploidy state of that chromosome.

In one aspect of this embodiment, DNA may be divided into from two to five fractions, from six to ten fractions, from ten to twenty fractions, or over twenty fractions. In another aspect the DNA may originate from a recently lysed cell.

In another aspect of this embodiment, the method may further include the step of amplifying the DNA in each of the fractions before it is genotyped in the genotyping step. In another aspect of this embodiment, the method may further include the step of diluting the DNA before dividing it into fractions. In one aspect of this embodiment, the DNA may be diluted down to from about 0.01 to about 0.2, from about 0.2 to about 0.4, or from about 0.4 to about 0.8 copies of the genome per well. In another aspect of this embodiment, the DNA may be diluted down to about equal to or less than one chromosome per well.

In another aspect of this invention, a Bayesian method may be used to determine the most likely ploidy state of the cell in the using step. In another aspect of this embodiment, the Well Frequency Distribution (WFD) or the Highest Well Frequency (HWF) method may be used to determine the ploidy state of the cell in the using step.

In some embodiments of the invention, the genetic matter from a single cell may be divided into a plurality of fractions, and the DNA in each of those fractions may be individually amplified and genotyped at a plurality of alleles. In this embodiment, the measured genotypes may be used to determine the ploidy state of the cell. Since the initial division of the DNA may have occurred when there was only one copy of DNA, the number of fractions in which a given allele is found may imply the minimum number of homologous chromosomes, from which that allele originated, that may have been present in the cell. The “well frequency,” or WF, is the number of wells (fractions) in which a given allele is detected. The highest well frequency (HWF) is the highest number of fractions that any alleles from a given chromosome are found. In this embodiment, the ploidy state at that chromosome may be taken to be the HWF of the alleles that are found on that chromosome. This method is referred to herein as the HWF method.

For the HWF method to give accurate results, the number of alleles that are measured on each chromosome may be sufficiently large that at least some alleles with the HWF corresponding to the ploidy state of that chromosome may be very likely to be measured. In one embodiment, at least 20 alleles may be measured on each chromosome. In one embodiment, at least 50 alleles may be measured on each chromosome. In another embodiment, at least 100 alleles may be measured on each chromosome. In another embodiment, at least 200 alleles may be measured on each chromosome.

In an example of the application of this method, the genetic material from one cell may be split into three fractions (N=3), and then the DNA in each of the fractions may be amplified and genotyped. If the cell is trisomic at one (or more) of those chromosomes, then each allele on the trisomic chromosome(s) may be present three times, and for each allele (assuming a 0% ADO rate), there may be a (N-1)(N-2)/N²= 2/9 chance that each of the three copies of the allele will end up in different fractions. By measuring a number of alleles, n, such that n may be much greater than five (for example N=25, 50 or 100) on each chromosome, it may be highly likely that at least some of the alleles on trisomic chromosomes may be detected in three different wells, and thus it may be possible to detect the trisomy with high accuracy. For disomic chromosomes, there may be a (1/N) possibility that two diploid copies of a given allele are in the same fraction, and a (N-1)/N=⅔ chance that the two copies of a given allele may be in different fractions. Provided that the number of alleles measured per chromosome may be adequate to determine trisomy with high accuracy, this method may also be able to determine disomy with high accuracy. Another way to increase the chances that the HWF is detected may be to increase the number of fractions (wells) into which the DNA is divided.

A high ADO rate may also decrease the accuracy of the method, as the chance of measuring at least one allele with the HWF for each chromosome will drop. One way to compensate for this problem may be to measure more alleles. Consider a non-zero ADO rate where the drop outs may be randomly distributed. The chance of both alleles of a homologous chromosome being detected in different fractions may be [(N-1)/N]*(1-ADO)², and in the case of a trisomic chromosome, the chance that all three alleles are detected in different fractions is [(N-1)(N-2)/N²]*(1-ADO)³. For the case where ADO is 50%, then the chance of a given allele having WF=2, in the case of a disomy may be ⅙ (⅔×¼), and the chance of a given allele having WF=3, in the case of a trisomy may be 1/36 ( 2/9×⅛). For example, in the case of a blastomere with trisomy 21, where 360 alleles from chromosome 21 are measured, and a 50% ADO rate is observed, one would expect that, on average, 45 of the alleles would have WF=0, 185 of the alleles would have WF=1, 120 of the alleles would have WF=2, and 10 of the alleles would have WF=3. The HWF of three may be detected, on average, for ten alleles.

The accuracy of the HWF method can also be compromised by a high ADI rate. One expects that for euploid cells, a given allele may be found in no more than two of the fractions. If genetic material from one or more alleles is found in three of the fractions, this implies that three homologous chromosomes containing that allele may have been present in the original cell, and thus it may be trisomic for the chromosome that corresponds to that allele. However, a non-zero ADI rate can result in a given allele being detected in more fractions than there were corresponding chromosomes in the original cell. For example, an ADI could result in a given allele being detected in three fractions when, in reality, the cell was euploid. In one embodiment, the ploidy state of a given chromosome may only be taken to be the HWF for the set of alleles found on that chromosome if the number of alleles with the HWF exceeds a threshold percentage of alleles.

Given a sufficiently large number of alleles on a given chromosome, combined with a sufficiently large number of fractions, it may be possible to determine the ploidy state, using this approach, with a high level of confidence. In one aspect of this embodiment, the number of alleles called may be more than about 20. In another aspect of this embodiment, the number of alleles called may be more than about 40. In one aspect of this invention, the number of fractions may be between two and five. In another aspect of this invention, the number of fractions may be more than about five. In another aspect of this invention, the number of fractions may be more than about ten. In another aspect of this invention, the number of fractions may be more than fifteen, more than twenty and/or more than twenty-five. In another embodiment of the invention, a Bayesian approach to computing may be used to compute the probability of each hypothesis given the data, either as a function of ADO rate and ADI rate, or integrated over all possible levels of ADO and ADI, and where each hypothesis corresponds to a given possible ploidy state. In one aspect of the invention, the ploidy state can be determined with greater than 90% confidence. In another embodiment of the invention, the ploidy state can be determined with greater than 99% confidence.

In another embodiment of the invention, the ploidy state of a cell at a given chromosome can be determined by looking at the distribution of the WFs of the set of alleles found on that chromosome, and comparing that distribution to expected distributions for different possible ploidy states. The distribution of the WF within the set of alleles on a given chromosome is called the well frequency distribution, or WFD. For example, if we measure ten alleles on chromosome 21, and if two different alleles are detected in two wells, five alleles are detected in one well only, and the remaining three alleles are not measured in any wells, we would say that the WFD (0:1:2)=(3:5:2). Different ploidy states give rise to different WFD for a given set of experimental conditions. The observed WFD for the allele in that set may be characteristic of a specific ploidy state, and can be used to determine the ploidy state of the cell. In one embodiment, the WFD for a set of alleles found on the same chromosome can be used to determine the ploidy state of that chromosome. This method is referred to herein at the WFD method.

The likelihood that a given allele may be detected in zero, one, two, three or more fractions depends on a number of factors, including the ploidy state of the chromosome on which the allele may be located, the number of fractions into which the genetic material may be divided, the ADO rate, the ADI rate, and the homogeneity of the mixing. By analyzing the measured genetic data from each individual well, it may be possible to reconstruct the factors, such as ADO and ADI that affect the WFD for a set of alleles, and calculate the expected WFD for different ploidy states. By comparing the measured distribution to the expected distributions calculated for different ploidy states, it may be possible to determine the most likely actual ploidy state of one or a plurality of chromosomes in the cell.

An advantage of the WFD method over the HWF method is that fewer allele calls per chromosome may be necessary to reach a given level of confidence. In some embodiments, the ploidy determination may be made by looking at the distribution of WF, and comparing it to an expected distribution of WFs, as calculated for the possible conditions (ADO, ADI, etc.). This may be because the WFD method uses the WF data from all of the alleles, while the HWF method only utilizes data from those alleles with the HWF; therefore when using the HWF method, more alleles must be measured to get a similar number of informational alleles.

A brief example is given here for the purposes of illustration. Take the above example, with a cell that is trisomic at chromosome 21, where the DNA was divided into three fractions, and amplified with an ADO rate of 50%. One would expect the WFD (0:1:2:3) for a trisomic cell to be, on average, in a ratio of (9:37:24:2), in the case of a euploid cell, one would expect the WFD to be (18:42:12:0), and for a monosomic cell, the WFD would be expected to be (36:36:0:0). The observed WFD is then compared to the set of expected WFD for the different possible ploidy states, and the ploidy state that is most likely to be statistically true is taken to be the correct ploidy state for that chromosome. In another embodiment of the invention, a Bayesian approach to computing is used to compute the probability of each hypothesis given the data, either as a function of ADO rate and allele drop in (ADI) rate, or integrated over all possible levels of ADO and ADI, and where each hypothesis corresponds to a given possible ploidy state. Other factors may have an impact on the distribution of the chromosomal fragments, and thus the wells in which alleles are measured; these can be taken into account without changing the essence of the invention.

In another embodiment of the invention, other forms of aneuploidy, such as nullsomy, monosomy, disomy, trisomy, and tetrasomy can be detected. In another embodiment, this method can detect uniparental disomy and mitotic trisomies where two (or more) chromosomes are genetically identical. This method may be in contrast to most methods that use genotyping to determine ploidy states. Additionally, this method can differentiate mitotic trisomies from disomies, and uniparental disomy from monosomies, which most methods are not able to do. In one aspect of the invention, this method is combined with other informatics based approaches, such as the Parental Support™ method, to identify the number and origin of all the chromosomes in a cell. In some embodiments of the invention, the methods disclosed herein may be used in the context of preimplantation genetic diagnosis during IVF. In some embodiments of the invention, the methods disclosed herein may be used in the context of prenatal diagnosis.

As opposed to the embodiments concerning haplotyping, the embodiments involving ploidy calling by dividing the single cell genetic material into a plurality of fractions may be more accurate when the chromosomal segments are fragmented such that each of the alleles on a chromosome are be divided into each fraction in a roughly statistical manner. Therefore, in the embodiments concerning ploidy calling, no special care may be taken to handle the DNA in a manner that will minimize strand breakage.

The accuracy of the method for ploidy determination, described herein, may increase as the number of fractions increases. With increasing fraction number, the chances that the individual alleles from homologous chromosomes may be detected in different fractions increases. Moreover, the distributions of WF for different ploidy states may become more distinctive as the number of fractions increases.

The specific methods described herein for dividing genetic material from a single cell into a plurality of fractions, amplifying and genotyping those fractions, in the context of determining the haplotype of that cell, may be applied in the context of determining the ploidy state. There are many methods that can be used to amplify and genotype genetic material originating from a single cell, some described herein, and any of those methods could be used in conjunction with, or as part of the method described here for the purpose of ploidy determination.

A person of ordinary skill in the art would recognize that given the benefit of this disclosure, various aspects and embodiments of this disclosure may implemented in combination or separately. For example, the invention includes the embodiment wherein the ploidy state, haplotype or whole genome amplification information may be used for the purpose of embryo selection during in-vitro fertilization or prenatal genetic diagnosis. Another embodiment of the present invention includes the combination of the methods and wherein the ploidy state, haplotype and/or whole genome amplification information may be generated from the same DNA sample. In an aspect of this embodiment, the DNA sample can also be used to generate genetic information on a target individual using the Parental Support™ method. In one aspect of this embodiment, the DNA sample may be a single cell.

In one aspect of any of the above embodiments, one or a plurality of parameters can be altered without changing the essence of the invention. For example, the genetic data may be obtained using any high throughput genotyping platform, it may be obtained from any genotyping method, or it may be simulated, inferred or otherwise known.

In one aspect of any of the above embodiments, it may be possible to use the methods disclosed herein in the context of cancer genotyping and/or ploidy determination, where one or more cancer cells may be considered the target individual, and the non-cancerous tissue of the individual afflicted with cancer may be considered to be the related individual. The non-cancerous tissue of the individual afflicted with the target could provide the set of genotype calls of the related individual that would allow chromosome copy number determination of the cancerous cell or cells using the methods disclosed herein.

In an aspect of any of the above embodiments of the invention, the techniques described for measuring genetic data are applied to the process of pre-implantation diagnosis during in vitro fertilization. In the above embodiments, it may be envisioned that the use of this method may facilitate diagnoses focusing on inheritable diseases, chromosome copy number predictions, increased likelihoods of defects or abnormalities, as well as making predictions of susceptibility to various disease-and non-disease phenotypes for individuals. According to some embodiments, the systems, methods, and techniques of the invention are used in methods to decrease the probability for the implantation of an embryo specifically at risk for a congenital disorder and/or a chromosome abnormality by testing at least one cell removed from early embryos conceived by in vitro fertilization and transferring to the mother's uterus those embryos determined not to have inherited the congenital disorder.

In an aspect of any of the above embodiments of the invention, the techniques described for measuring genetic data may be applied to the process of prenatal diagnosis in conjunction with amniocentesis, chorion villus biopsy (CVB), fetal tissue sampling, or other non-invasive prenatal diagnosis. In the above embodiments, it may be envisioned that the use of these methods may facilitate diagnoses focusing on inheritable diseases, chromosome copy number predictions, increased likelihoods of defects or abnormalities, as well as making predictions of susceptibility to various disease-and non-disease phenotypes for individuals. Any of the embodiments detailed above can be used for prenatal diagnosis at an early stage of pregnancy. In one aspect of this embodiment, the prenatal diagnosis using the above methods can be done before about 14 weeks of gestation. In another aspect of the invention, the prenatal diagnosis can be performed between about ten weeks of gestation to about fourteen weeks of gestation. In another aspect of the invention, the prenatal diagnosis can be performed before about ten weeks of gestation. In aspect of this embodiment, DNA from the fetus can be collected from the material blood for analysis in the above methods.

In one aspect of any of the above embodiments, the congenital disorder may be a malformation, neural tube defect, chromosome abnormality, Down's syndrome (or trisomy 21), Trisomy 18, spina bifida, cleft palate, Tay Sachs disease, sickle cell anemia, thalassemia, cystic fibrosis, Huntington's disease, and/or fragile x syndrome. Chromosome abnormalities include, but are not limited to, Down syndrome (extra chromosome 21), Turner Syndrome (45X0) and Klinefelter's syndrome (a male with 2 X chromosomes). In one embodiment, the malformation may be a limb malformation. Limb malformations include, but are not limited to, amelia, ectrodactyly, phocomelia, polymelia, polydactyly, syndactyly, polysyndactyly, oligodactyly, brachydactyly, achondroplasia, congenital aplasia or hypoplasia, amniotic band syndrome, and cleidocranial dysostosis. In one aspect of this embodiment, the malformation may be a congenital malformation of the heart. Congenital malformations of the heart include, but are not limited to, patent ductus arteriosus, atrial septal defect, ventricular septal defect, and tetralogy of fallot. In another aspect of this embodiment, the malformation may be a congenital malformation of the nervous system. Congenital malformations of the nervous system include, but are not limited to, neural tube defects (e.g., spina bifida, meningocele, meningomyelocele, encephalocele and anencephaly), Arnold-Chiari malformation, the Dandy-Walker malformation, hydrocephalus, microencephaly, megencephaly, lissencephaly, polymicrogyria, holoprosencephaly, and agenesis of the corpus callosum. In another aspect of this embodiment, the malformation may be a congenital malformation of the gastrointestinal system. Congenital malformations of the gastrointestinal system include, but are not limited to, stenosis, atresia, and imperforate anus.

According to some embodiments, the systems, methods, and techniques of the invention may be used in methods to increase the probability of implanting an embryo obtained by in vitro fertilization that is at a reduced risk of carrying a predisposition for a genetic disease. In another embodiment, the methods and techniques of the invention may be used to determine the probability of a fetus having a predisposition for a genetic disease. In one aspect of this embodiment, the genetic disease may be either monogenic or multigenic. Genetic diseases include, but are not limited to, Bloom Syndrome, Canavan Disease, Cystic fibrosis, Familial Dysautonomia, Riley-Day syndrome, Fanconi Anemia (Group C), Gaucher Disease, Glycogen storage disease 1a, Maple syrup urine disease, Mucolipidosis IV, Niemann-Pick Disease, Tay-Sachs disease, Beta thalessemia, Sickle cell anemia, Alpha thalessemia, Beta thalessemia, Factor XI Deficiency, Friedreich's Ataxia, MCAD, Parkinson disease-juvenile, Connexin26, SMA, Rett syndrome, Phenylketonuria, Becker Muscular Dystrophy, Duchennes Muscular Dystrophy, Fragile X syndrome, Hemophilia A, Alzheimer dementia-early onset, Breast/Ovarian cancer, Colon cancer, Diabetes/MODY, Huntington disease, Myotonic Muscular Dystrophy, Parkinson Disease-early onset, Peutz-Jeghers syndrome, Polycystic Kidney Disease, Torsion Dystonia, and other genetic diseases or disorders.

In one embodiment of the invention, one or more of the disclosed methods may be employed in conjunction with other methods, such as the Parental Support™ method, to determine the genetic state of one or more embryos for the purpose of embryo selection in the context of IVF, or for prenatal diagnosis. This may include the harvesting of eggs from the prospective mother and fertilizing those eggs with sperm from the prospective father to create one or more embryos. It may involve performing embryo biopsy to isolate a blastomere from each of the embryos. It may involve amplifying and genotyping the genetic data from each of the blastomeres, or analyzing fetal genetic material isolated from the maternal blood, CVB, amniocentesis or other method. It may involve amplifying and genotyping the genetic data of a blastomere or fetal genetic material using the spike-in method described herein. It may include obtaining, amplifying and genotyping a sample of diploid genetic material from each of the parents, as well as one or more individual sperm from the father. It may involve determining the genetic haplotypes of the blastomere, or of the genetic material of related individuals using the methods described herein. It may involve incorporating the measured diploid and haploid data of both the mother and the father, along with the measured genetic data of the embryo of interest into a dataset. It may involve using one or more of the statistical methods disclosed in this patent to determine the most likely state of the genetic material in the embryo given the measured or determined genetic data. It may involve the determination of the ploidy state of the embryo of interest using the measured diploid genotype, and an informatics based approach such as PS. It may involve the determination of the ploidy state of the embryo of interest using the distribution of alleles that are detected in a plurality of fractions, each fraction having been created by dividing the genetic material from a single cell prior to amplification and genotyping. It may involve the determination of the presence of a plurality of known disease-linked alleles in the genome of the embryo. It may involve making phenotypic predictions about the embryo. It may involve generating a report that is sent to the physician of the couple so that they may make an informed decision about which embryo(s) to transfer to the prospective mother.

### Example 1

### Sigma WGA Kit Spike-In Protocol

Single tissue culture cells (AG16777, Coriell) were sorted through five 1× PBS droplets by mouth pipette and placed in 5 μL Lysis and Fragmentation buffer (according to Sigma's instructions), and the reactions incubated at 50° C. for 1 hour, 99° C. for 4 min, and placed on ice. A 2 μL Library Preparation mix was added composed of 1 μL 1× Single Cell Library Preparation Buffer (Sigma), 0.5 μL Library Stabilization Solution (Sigma), and 0.5 μL Library Preparation Enzyme (Sigma). Reactions were incubated according to Sigma. A 30 μL Amplification mix was then added composed of 3.75 μL 10× Amplification Master Mix (Sigma), 2.5 μL WGA DNA Polymerase (Sigma), 1 μL 10 μM each of the two spike-in primers (in the multiplex spike-ins; 1 μL of a 0.3 nM primer pool was added), and 23 μL H₂O. Reactions were incubated according to Sigma. Five microliters of the solution was loaded on a 1.5% agarose gel (to verify WGA efficiency) while another 5 μL products were diluted 1/15 and 1/50 by addition of 70 and 245 μL H₂O, respectively.

For the PCR based analysis, 1 μL of 1/50 diluted WGA products were added to 9 μL Advantage 2 (Clontech) PCR mix containing 2 pmol each of the spiked-in primers. Reactions were placed in a thermocycler and incubated at 95° C. for 3 min, followed by 34 cycles of 95° C. for 30 sec and 65° C. for 40 sec. 8 μL of the solution was then loaded on 1.5% agarose gel (0.7×SYBR Green I, Invitrogen). Absence of amplification product was categorized as locus dropout (LDO).

For TAQMAN analysis, 2.5 μL of 1/15 diluted MDA products were combined with 2.5 μL TAQMAN® Universal PCR Master Mix (ABI) and TAQMAN primer/probe mix (20×, ABI), according to manufacturer, and analyzed in a ABI 7900 instrument. Multiplex spike-ins (with 8 primer pairs immediately spanning their SNP, respectively) were analyzed using 1×BioRad MasterMix, 1 82 l 1/50 diluted Amplification products, and 2 μL 2.5 μM (each) primer mix in total 6 μl, and results verified with Dissociation Curve analysis.

### Results

The results are summarized below from single cell spike-in of six primer pairs either individually (Table 2) or in multiplex (Table 3). The data in Table 2 shows that the use of a single spike-in primer lowers the LDO rate from 79% to 7%. The data in Table 3 shows that the use of multiple spike-in primers (in this case 7 or 8) lowered the LDO rate from 29% to 9%. Note that TAQMAN was used to measure the LDO rate of the non-targeted alleles to ensure that the addition of the spike-in primers did not adversely affect the ADO rate of the non-targeted alleles.

TABLE 2
Summary of results from single cell spike-in of single locus
specific primers based on agarose gel analysis and TAQMAN.
Data is presented in the format (a/b), where ‘a’ is the number
of alleles displaying LDO and ‘b’ is the number of alleles tested.
Data for non-target alleles were derived from TAQMAN
analysis of non-related SNPs. *Two samples had smeared PCR
products spanning the correct size.

| LDO (#/total cells) | |
|---|---|
| No spike-in | Single spike-in |

| | Target | Nontarget | Target | Nontarget |
|---|---|---|---|---|
| Locus | alleles | alleles | alleles | alleles |
| 13096 | 6/7 | 16/28 | 0/7 | 5/14 |
| 2668640 | 7/7 | 16/28 | 0/7 | 3/14 |
| 2602208 | 2/7 | 4/14 | 0/7 | 0/7 |
| 656642 | 5/7 | 1/14 | 0/7 | 0/7 |
| 656763 | 6/7 | 6/14 | 3/7 | 0/7 |
| 2540897 | 7/7 | 4/14 | 0/7 | 0/7 |
| Total | 33/42 | 47/112 | 3/42 | 8/56 |
| | 79% | 42% | 7% | 14% |

TABLE 3
Summary of results from single cell WGA with multiplex
spike-ins based on SYBR analysis on an ABI 7900
instrument. Data is presented in the format (a/b), where
‘a’ is the number of alleles displaying LDO and ‘b’ is the
number of alleles tested. Data for non-target alleles were
derived from TAQMAN analysis of non-related SNPs.

LDO in Multiplex spike-in,
Invitrogen primer pools

| | | Pool A | Pool B | Pool C |
|---|---|---|---|---|
| | No spike-in | (8-plex) | (8-plex) | (7-plex) |
| Target 1 | 5/19 | 0/7 | 0/6 | 0/7 |
| Target 2 | 5/19 | 0/7 | 3/6 | 0/7 |
| Target 3 | 8/19 | 1/7 | 2/6 | 1/7 |
| Target 4 | 5/19 | 1/7 | 0/6 | 4/7 |
| Target 5 | 8/19 | 0/7 | 0/6 | 1/7 |
| Target 6 | 4/19 | 0/7 | 0/6 | 0/7 |
| Target 7 | 4/19 | 1/7 | 0/6 | 0/7 |
| Target 8 | 3/13 | 0/7 | 0/6 | N/A |
| Total | 42/146; | 3/56; | 5/48; | 6/49; |
| | 29% | 5% | 10% | 12% |
| (A + B + C) | | 14/153; 9% | | |
| Nontarget | 55/270; | 23/91; | 8/78; | 41/112; |
| loci | 20% | 25% | 10% | 37% |

### Example 2

### GE MDA Kit Spike-In Protocol

Single tissue culture cells (GM11392, Coriell) were sorted through five 1× PBS droplets by mouth pipette and placed in 4 μL lysis buffer (Proteinase K in Reconstitution buffer (Arcturus)) supplemented with 0.5 μL 0.1 M DTT, 0.3 μL 1M KCl, 0.24 μL 25 mM MgCl₂, 0.06 μL Tris-HCl (pH 7.5), and 1 μL 5 uM primers (each, of forward and reverse). Reactions were incubated at 56° C. for 1 hour, 95° C. for 10 min, 25° C. for 15 min, and then placed on ice. A 26.1 μL MDA mix was added composed of 12 μL Sample buffer (GE), 12 μL Reaction buffer (GE), 1.2 μL Enzyme Mix (GE), 0.9 μL BSA (10 μg/μl). Reactions were incubated at 30° C. for 2 hours, followed by 95° C. for 5 min, and finally diluted 1/15 by adding 450 μL H₂O.

For the PCR based analysis, 1 μL of 1/15 diluted MDA products were added to 9 μL Advantage 2 (Clontech) PCR mix containing 2 pmol each of the spiked-in primer pair (note; all six primer pairs was used in the multiplex spike-in, but each pair was then used separately in this 2^{nd} PCR). Reactions were placed in a thermocycler and incubated at 95° C. for 3 min, followed by 34 cycles of 95° C. for 30 sec and 65° C. for 40 sec. 8 μL of the solution was then loaded on 1.5% agarose gel (0.7×SYBR Green I, Invitrogen). Absence of amplification product was categorized as locus dropout (LDO).

For TAQMAN analysis, 2.5 μL of 1/15 diluted MDA products were combined with 2.5 μL TAQMAN® Universal PCR Master Mix (ABI) and TAQMAN primer/probe mix (20×, ABI), according to manufacturer, and analyzed in a ABI 7900 instrument.

Alternative Lysis Methods that Can be Used:

In the Alkaline lysis, 2.5 μL of Alkaline lysis buffer (200 mM KOH, 50 mM DTT) were used, 0.5 μL of 5 μM each spike-in primer (Fw+Rev) for a total volume of 3.0 μL. The lysis solution was neutralized with a solution made up of 2.5 μL Neutralization buffer (900 mM Tris, 300 mM KCl, 200 mM HCl). After adding, the total volume is 5.5 μl. The samples were kept at 25° C. for 15 min, then on ice, and proceed to MDA.

In the ‘New PK’ lysis, it was found to be beneficial to perform a two-step addition of spike-in/MDA reagents, in order for the primers to hybridize before the MDA hexamers are introduced. Therefore, 4.00 μL M-PER (Piercenet, #78503), 0.20 μL 2.5M KOH, 0.05 μL 1M DTT, 0.75 μL Proteinase K (Sigma, #P4850), and 1.00 μL of a 5 μM solution was mixed in each spike-in primer (Fw+Rev) for a total volume of 6.1 μL. The cells were left to lyse at 37° C. for 30 min (or in some cases longer), and then heated to 85° C. for 5 min, before returning to room temperature. Then 0.35 μL 1M KCl, 0.28 μL 50 mM EDTA, and 0.62 μL 1M Tris-HCl pH 7.6 (Sigma, #T2788) were added, for a total volume of 7.35 μL. This mixture was allowed to sit at 25° C. (rt) for 15 min, then on ice. In the final step, 40 μL MDA mix was added.

### Results

Table 4 summarizes the results from single cell spike-in of six primer pairs either individually or in multiplex. The data show that the use of a single spike-in primers lowers the LDO rate from 26% to 7%, and that the use of multiplex spike-in primers (in this case 6) lowered the ADO rate from 26% to 8%. Note that TAQMAN was used to measure the ADO rate of the non-targeted alleles to ensure that the addition of the spike-in primers did not adversely affect the ADO rate of the non-targeted alleles.

Note that in all of the spike-in methods disclosed here, the maximum number of targeted primers that can be run without further optimization is typically from five to ten. In some cases, as many as 20 or even 100 could be run with additional optimization of conditions, or as technological advances allow.

TABLE 4
Summary of results from single cell spike-in of locus specific primers
based on agarose gel analysis (No spike-in; see Table 3).

| | LDO (#/total cells) | | | | | |
|---|---|---|---|---|---|---|
| | | | | | Multiplex spike-in | |
| | No spike-in | | Single spike-in | | (6-plex) | |
| | | Non | | Non | | Non |
| | Target | target | Target | target | Target | target |
| Locus | loci | loci | loci | loci | loci | loci |
| 13096 | 7/21 | | 1/15 | | 1/14 | |
| 2668640 | 6/21 | | 0/14 | | 0/14 | |
| 2602208 | 6/21 | | 1/15 | | 2/14 | |
| 656642 | 4/21 | | 2/14 | | 2/14 | |
| 656763 | 6/21 | | 2/13 | | 1/14 | |
| 2540897 | 4/21 | | 0/13 | | 1/14 | |
| Total | 33/126; 26% | 33% | 6/84; 7% | 40% | 7/84; 8% | 33% |

Data is presented in the format (a/b), where ‘a’ is the number of alleles displaying LDO and ‘b’ is the number of alleles tested. Data for non-target alleles were derived from TAQMAN analysis of non-related SNPs.

### Example 3

### Single Cell Haplotyping

Single 16777, 16778, and 16782 (Coriell) cells were sorted through five PBS washes and placed in 5 μL lysis buffer containing 4 μL M-PER (Piercenet), 0.2 μL 2.5 M KOH, 0.05 μL 1M DTT, and 0.75 μL Proteinase K (Sigma). The lysis reaction was incubated at 37° C. for 10 minutes, followed by 75° C. for 4 min, and then placed on ice. Incubation times may vary depending on cell type. A 115 μL MDA mix composed of 50 μL Sample Buffer (GE), 50 μL Reaction Buffer (GE), 5 μL Enzyme Mix (GE), 4 μL BSA (10 μg/μl), and 6 μL 10× PBS, was added at room temperature according to the details above, and the reaction left at room temperature for five minutes. Using the same pipette tip, 24 μL was transferred to each of four tubes (leaving approximately 24 μL in the original tube). All reactions were incubated at 30° C. for 2 hours, then 75° C. for 10 min. Note that this is a deviation from the MDA protocol; its purpose here is to preserve integrity of the products to allow analysis by the ILLUMINA INFINIUM assay, followed by ice. Other variations of the protocol may be used while preserving the essence of the invention.

If an ILLUMINA INFINIUM array is used for analysis, most of each reaction volume may be dedicated to that, while a fraction (for example 4 μl) may be diluted (at least 1/10) for TAQMAN analysis, which may then serve as verification that each reaction does in fact contain MDA products. Other ways of analyzing the haplotypes are possible, for example real-time SYBR PCR, TAQMAN, etc.

The TAQMAN analysis performed here included probes targeting SNPs located on chromosomes 7 and X, but any appropriately chosen probes may work. However, having several probes targeting different chromosomes may indicate whether or not the dilution (by the 115 μL MDA mix) was efficient by the appearance of hits in different reactions. For example, if all hits, independent of chromosome, were found in the same reaction, that could imply that the experiment will be uninformative because the entire genome may be located in that reaction.

TAQMAN results showed that haplotype information about SNPs distanced more than 1.6 Mb apart could be obtained. Both TAQMAN and microarray data show that it is possible to obtain haplotypes of up to 10 Mb using this method. Further optimization of this method is expected to allow haplotypes of up to 25 Mb to be measured.

### Example 4

### PCR Haplotyping

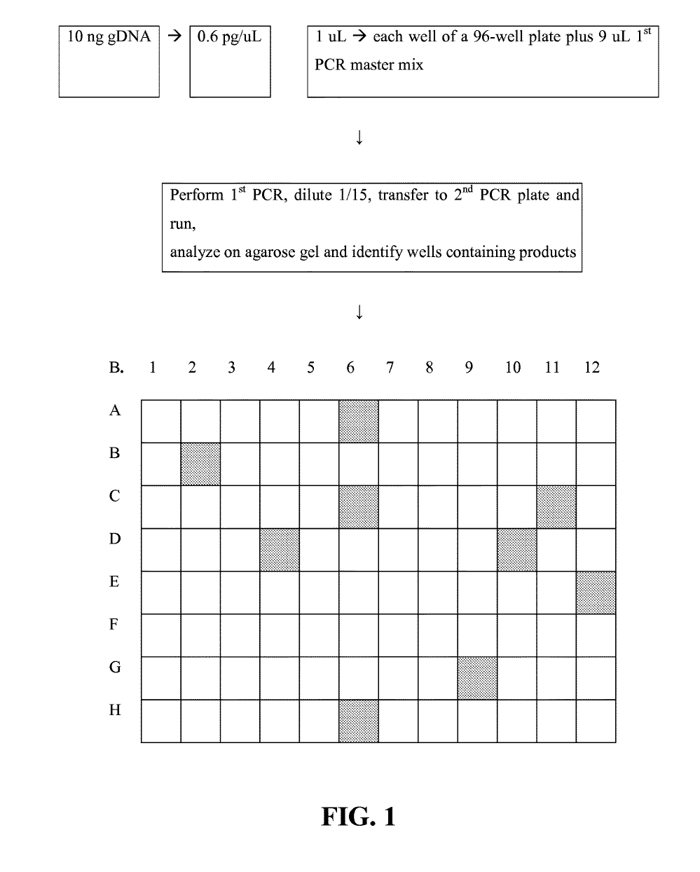

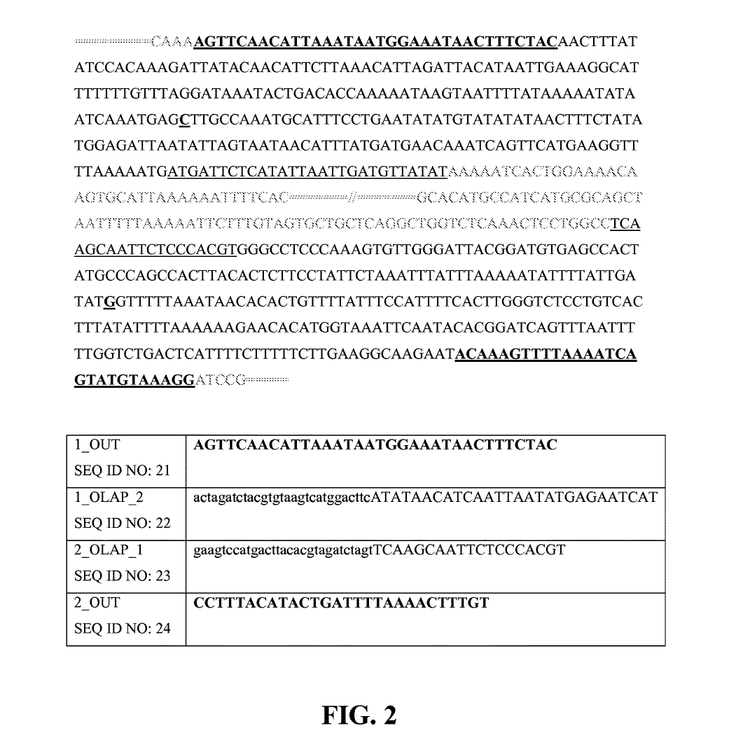

FIG. 1 shows a flow chart illustrating some steps of the method for PCR haplotyping. FIG. 1 details a gDNA being diluted to 0.6 pg/μl and 1 μL is placed in each well of a 96-well plate; followed by a 1^{st} PCR with two primer pairs (see FIG. 2) is performed followed by a 1/15 dilution. Samples from the 1^{st} PCR dilution is transferred to corresponding wells of a 2^{nd} 96-well PCR with only the outer primers (see FIG. 2). After completion, a fraction from each reaction is loaded to an agarose gel. Wells that contains product of correct size are identified (grey color) and their content purified by a PCR purification kit and finally Sanger sequenced.

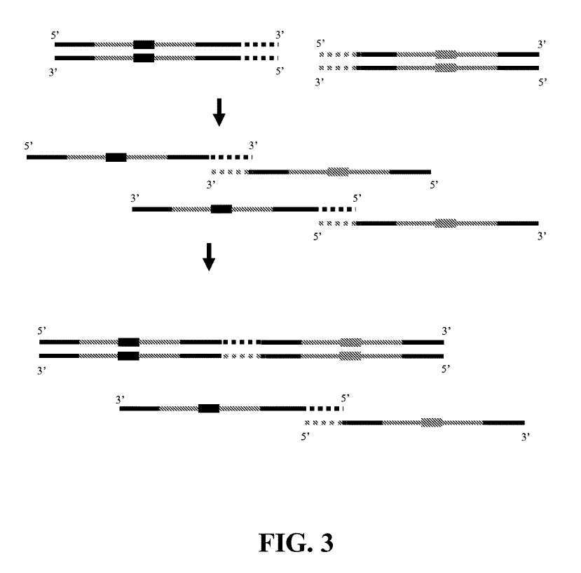

In FIG. 2, grey sequences indicate non-targeted regions, underlined sequence indicate primer sequences where bold indicate outer primer sequences. The interrupted grey sequence represents non-targeted regions and can be of any realistic length. Below the spike-in primer design are the four primers (SEQ ID NOs: 21-24) used for this minichromosome, where non-capitalized sequences are overlapping sequence that will connect the two individual PCR products. FIG. 3 uses black lines to indicate the individual PCR primers, grey lines to show the sequence surrounding the SNPs (black and grey boxes), and dotted lines to represent the overlapping sequences, respectively. The upper panel represents the two individual PCR products, the middle panel the two possible hybrids, and the lower panel the resulting minichromosome (full-length PCR product) and the remaining non-reacted DNA strands.

Initially, genomic DNA (gDNA) from either whole blood, cell culture or other source is gently diluted in a suitable buffer. Note that the preparation method used here plays an important part for the integrity of the gDNA; a method is chosen which is least harmful for integrity. For the experiments described herein, intact cells were used as starting material—no preparation of gDNA was performed). Suitable media for dilution is saline buffers (e.g. 1× PBS) or Tris-HCl of pH 7-8 that may contain 1 mM EDTA (to bind divalent cations), 1% EtOH (to reduce free radicals), or Salmon Sperm DNA (SS-DNA, to block binding of genomic DNA to tube walls). Mixing was performed extremely gently, e.g. by inverting the tube/vial slowly, or even by leaving the tube/vial at room temperature for some time (e.g. 5-15 min).

Fractions of the diluted gDNA were then distributed to several reaction wells (e.g. a 96-well plate) so that the likelihood that any given well will contain two diploid copies was small. Calculations of this approach are described in Konfortov et al.

Each fraction was then combined with a PCR mastermix containing four (or more) PCR primers according to Wetmur et al. The primers are designed to amplify regions spanning two (or more) SNPs located within a certain range of each other. The length limits have not been determined and success depends on integrity of the gDNA, however, in theory the SNPs could be separated by any realistic length.

The primers closest to the targeted region(s) contain an additional reverse complementary sequence on their 5′ ends (lower case letter in FIG. 2), so that the sense and anti-sense strands of each PCR product can hybridize to each other, respectively (FIG. 3), thereby forming a “minichromosome” when made double stranded. Thus, the minichromosome contains sequence from the two (or more) targeted regions combined into one PCR product. If more than two SNPs are targeted, both primers of the middle SNP(s) needs to contain additional reverse complementary sequences in order to connect (by hybridization) to its closest neighbor. The formation of the minichromosome may take place during the initial PCR, or during a second PCR, depending on the relative concentrations of the primers. Higher relative concentration of the “outer” primers tend to drive minichromosome formation, however, it was found that equal concentration of all primers produced the highest amount of both individual products, which in the second PCR produced higher yields of minichromosomes.

Following the strategy of equal primer concentration, a first PCR is run after which the products may be analyzed on agarose gel, the result being the individual PCR products, or if minichromosome formation did take place, additional products of size corresponding to the combined sizes of the individual PCR products, less half of the additional sequences (dotted lines in FIG. 3). All reactions are diluted by addition of H₂O (irrespective of whether minichromosomes were formed or not).

A fraction of the diluted products is then transferred to a second PCR containing only the outer primers for the two SNPs (the ones most distal to each other). After PCR completion, a sample from each reaction is loaded on an agarose gel, and reactions with products of sizes corresponding to the combined sizes of the individual PCR products, less half of the overlapping sequences, are identified. The remaining content of those reactions are purified (e.g. with a PCR purification kit) and the sequence determined by Sanger sequencing (using one or both of the outer primers) or by other means, such as TAQMAN, thus giving the phase of the two SNPs. By definition, an informative haplotype is obtained if the following criteria are met: the two (or more) targeted SNPs are heterozygous (otherwise there is no point including them), and the obtained Sanger sequences are not in conflict with each other. For example, for the SNPs A/G and T/C (thus, the DNA investigated is heterozygous in both), the haplotype results should read A-T and G-C, or A-C and G-T.

The accuracy of the method for haplotype determination, described herein, increases as the number of fractions increases. With increasing fraction number, the chances that each individual section of DNA will be located in a different fraction from its homologous section, found on the homologous chromosome, thus increasing the potential that individual haplotypes are measured. The drawback to increasing the number of fractions is that for each fraction a separate genotyping is required, and increasing the number of fractions increases the cost of the method due to the cost of the genotyping microarray. Thus an optimal number of fractions may be found that balances the costs and benefits of increasing the number of fractions.

In the following experiment, approximately 1,000 GM08586 (Coriell) cells were lysed and used to create a 10 mL dilution containing 10 mM Tris-HCl (pH 7.5) and 10 μg/μl SS-DNA. Alternatively, prepared genomic DNA from cell culture could be used without lysis. The gDNA dilution was mixed gently by inverting the tube during approximately 5 min. A 1 mL PCR mastermix containing 60 pg from the gDNA dilution was prepared, and also mixed by gently inversion during 5 min. Ten microliters of the solution was deposited in each well of a 96-well plate, thus resulting in 0.6 pg/well. PCR primers are given in Table 5, and were designed to amplify a 300 bp region spanning SNPs rs10487377, rs2067080, and rs13224934 (NCBI annotation), separated by 7,802 bp and 3,493 bp, respectively. These SNPs had previously been genotypes as all heterozygous in this cell line by Sanger sequencing. Thus, the two most distal SNPs were separated by 11,295 bp. Other experiments with SNPs located further apart from each other showed little or no success, indicating that the assay failed to receive the intact target strand into the same reaction well (thus gDNA was too fragmented).

TABLE 5
PCR primer sequence used herein,
in the 5′ to 3′ direction.

| | |
|---|---|
| 680F_haplo | TTACCACCTTGCACACATTCATT |
| SEQ ID NO: 13 | |
| 681F_haplo | AAATTCCCCAGGACATTAAGAGC |
| SEQ ID NO: 14 | |
| 681R_haplo | TGCTTTCAAACTGTTGACATTCC |
| SEQ ID NO: 15 | |
| 682R_haplo | GGTGAGTTTGGAGACCTCAAATG |
| SEQ ID NO: 16 | |
| 680R_haplo_olap | ACTAGATCTACGTGTAAGTCATGGACTTCGGTT |
| SEQ ID NO: 17 | GGTTAATCTGTGCTCCAGT |
| 681F_haplo_olap | GAAGTCCATGACTTACACGTAGATCTAGTAAAT |
| SEQ ID NO: 18 | TCCCCAGGACATTAAGAGC |
| 681R_haplo_olap | ACTAGATCTACGTGTAAGTCATGGACTTCTGCT |
| SEQ ID NO: 19 | TTCAAACTGTTGACATTCC |
| 682F_haplo_olap | GAAGTCCATGACTTACACGTAGATCTAGTAGAA |
| SEQ ID NO: 20 | TCATCTTTTGCTCCAGAGG |

After the PCR, each well received 140 μL, H₂O for a 1/15 dilution, and 2 μL from each reaction was transferred to a 2^{nd} PCR well (maintaining the reaction plate coordinates), already containing 8 μL PCR mastermix with only the outer PCR primers. After this PCR, 2 μL of each reaction was analyzed on a 1.5% agarose gel, and wells containing PCR products of approximately 600 bp were identified, and their content purified by a PCR purification kit (Qiagen). Of the 680-681 plate (see Table 5), 12 wells were identified as containing the correct size product, and of the 681-682 plate, six wells were identified. The Sanger sequencing results are shown in Table 6 where only results with a single sequence trace were considered. From these data it was established that the rs10487377-rs2067080-rs13224934 haplotype of the GM08586 cell line was G-C-C or A-T-T.

TABLE 6
Sanger sequencing result from selected minichromosomes
identified after 2^{nd} PCR.

| | 680 | 681 | Note |
|---|---|---|---|
| 1. | G | C | |
| 2. | A | T | |
| 3. | A | C/T | Trace of G in 680 |
| 4. | G/A | C/T | |
| 5. | G | C | Trace of T in 681 |
| 6. | G/A | C/T | |
| 7. | A | T | Noisy |
| 8. | G | C | |
| 9. | A | T | Trace of C in 681 |
| 10. | G | C | Trace of other |
| | | | allele in both |
| 11. | G | C | |
| 12. | A | T | |
| | 681 | 682 | Note |
| 1. | C | C | Noisy in 682 |
| 2. | T | T | Noisy in 682 |
| 3. | T | T | |
| 4. | — | — | |
| 5. | — | — | |
| 6. | C | C/T | Trace of T in 681 |

### Ploidy Calling by Division

First, sort a single cell (or a plurality of cells in parallel but separately) through five PBS washes and place it in 5 μL lysis buffer containing 4 μL M-PER (Piercenet), 0.2 μL 2.5M KOH, 0.05 μL 1M DTT, and 0.75 μL Proteinase K (Sigma). Incubate the lysis reaction at 37° C. for 10 minutes, followed by 75° C. for 4 min, and then place the samples on ice. Incubation times may vary depending on cell type. Add a 115 μL MDA mix composed of 50 μL Sample Buffer (GE), 50 μL Reaction Buffer (GE), 5 μL Enzyme Mix (GE), 4 μL BSA (10 μg/μl), and 6 μL 10× PBS, at room temperature according to the details above, and leave the reaction at room temperature for five minutes. Using the same pipette tip, transfer 40 μL of the solution to each of three tubes. Incubate all reactions at 30° C. for 2 hours, 75° C. for 10 min, followed by ice. Use an ILLUMINA INFINUM genotyping array to determine the genotypes present in each fraction; follow the protocol suggested by the manufacturer. Other variations of the protocol may be used while preserving the essence of the invention.

If an ILLUMINA INFINIUM array is used for analysis, most of each reaction volume may be dedicated to that, while a fraction (for example 4 μl) may be used for TAQMAN analysis, which may then serve as verification that each reaction does in fact contain MDA products. Other ways of analyzing the alleles present in each fraction are possible, for example real-time SYBR PCR. Having several probes targeting different chromosomes may indicate whether or not the dilution (by the 115 μL MDA mix) was efficient by the appearance of hits in different reactions. For example, if all hits, independent of chromosome, are found in the same reaction, this could imply that the experiment will be uninformative because the entire genome may be located in that reaction.

For each of one hundred and forty four two SNPs on each chromosome, determine in how many fractions that allele is detected. Also determine the ADO rate, and the ADI rate. Assume the case that the ADO rate is 50%, and the ADI rate is negligible (<2%). If any allele from a given chromosome is detected in more than two wells, assume trisomy. To take into account the possibility of a non-zero ADI rate, one may wish to set a cut-off threshold for calling trisomy at two or three alleles detected in more than two fractions. If no alleles are detected in more than two fractions, but some alleles detected in two wells, then assume disomy. If no alleles are detected in more than one fraction, assume monosomy.

An alternate method to determining the ploidy state from the genotypic data measured from the fractions follows. Calculate the expected distribution of WF for monosomy, disomy, and trisomy for the ADO and ADI rate observed in the genotyping. Assuming an ADO rate of 50%, and ADI rate of 0%, and when measuring 144 alleles, the expected WFD (0:1:2:3) is (72:72:0:0) for monosomy, (36:84:24:0) for disomy, and (18:74:48:4) for trisomy. Use a Bayesian (or other statistically based) analysis to determine which of the distributions most likely predicts the correct ploidy state for each of the chromosomes.

### Laboratory Techniques

There are many techniques available allowing the isolation of cells and DNA fragments for genotyping, as well as for the subsequent genotyping of the DNA. The system and method described here can be used in conjunction with any of these techniques, and in many contexts, specifically those involving the isolation of fetal cells or DNA fragments from maternal blood, or blastomeres from embryos in the context of IVF. This description of techniques is not meant to be exhaustive, and it should be clear to one skilled in the art that there are other laboratory techniques that can achieve the same ends.

### Isolation of Cells

Adult diploid cells can be obtained from bulk tissue or blood samples. Adult diploid single cells can be obtained from whole blood samples using FACS, or fluorescence activated cell sorting. Adult haploid single sperm cells can also be isolated from a sperm sample using FACS. Adult haploid single egg cells can be isolated in the context of egg harvesting during IVF procedures.

Isolation of the target single cell blastomeres from human embryos can be done using techniques common in in vitro fertilization clinics, such as embryo biopsy. Isolation of target fetal cells in maternal blood can be accomplished using monoclonal antibodies, or other techniques such as FACS or density gradient centrifugation.

DNA extraction also might entail non-standard methods for this application. Literature reports comparing various methods for DNA extraction have found that in some cases novel protocols, such as the using the addition of N-lauroylsarcosine, were found to be more efficient and produce the fewest false positives.

### Amplification of Genomic DNA

Amplification of the genome can be accomplished by multiple methods including (but not limited to): Polymerase Chain Reaction (PCR), ligation-mediated PCR (LM-PCR), degenerate oligonucleotide primer PCR (DOP-PCR), Whole Genome Amplification (WGA), multiple displacement amplification (MDA), allele-specific amplification, various sequencing methods such as Maxam-Gilbert sequencing, Sanger sequencing, parallel sequencing, sequencing by ligation. The methods described herein can be applied to any of these or other amplification methods while keeping the essence of the invention unchanged.

Background amplification may be a problem for each of these methods, since each method would potentially amplify contaminating DNA. Very tiny quantities of contamination can irreversibly poison the assay and give false data. Therefore, it may be important to use clean laboratory conditions, wherein pre- and post-amplification workflows are completely, physically separated. Clean, contamination free workflows for DNA amplification are now routine in industrial molecular biology, and simply require careful attention to detail.

### Genotyping Assay and Hybridization

The genotyping of the amplified DNA can be done by many methods including (but not limited to): molecular inversion probes (MIPs) such as Affymetrix's GENFLEX TAG ARRAY, microarrays such as Affymetrix's 500K array or the ILLUMINA BEAD ARRAYS, or SNP genotyping assays such as Applied Bioscience's TAQMAN assay, other genotyping assays, or fluorescent in-situ hybridization (FISH). The Affymetrix 500K array, MIPs/GENFLEX, TAQMAN and ILLUMINA assay all require microgram quantities of DNA, so genotyping a single cell with either workflow would require some kind of amplification. Each of these techniques has various tradeoffs in terms of cost, quality of data, quantitative vs. qualitative data, customizability, time to complete the assay and the number of measurable SNPs, among others.

### Genetic Sources

The source of the genetic material used in the invention disclosed herein can be from any cell containing a nucleus or from any DNA with a known or suspected origin, including (but not limited to): one or more diploid cells from the target individual, one or more haploid cells from the target individual, one or more blastomeres from the target individual, extra-cellular genetic material found on the target individual, extra-cellular genetic material from the target individual found in maternal blood, cells from the target individual found in maternal blood, genetic material known to have originated from the target individual, and combinations thereof.

All patents, patent applications, and published references cited herein are hereby incorporated by reference in their respective entireties. It will be appreciated that several of the above-disclosed and other features and functions, or alternatives thereof, may be desirably combined into many other different systems or applications. Various presently unforeseen or unanticipated alternatives, modifications, variations, or improvements therein may be subsequently made by those skilled in the art which are also intended to be encompassed by the following claims.

1. A method of performing genome amplification on a nucleic acid sample from a subject, the method comprising:
adding one or more spike-in primers to a nucleic acid sample that targets one or more loci of interest; and
amplifying the nucleic acid sample using a method for whole genome amplification,
wherein the addition of the one or more spike-in primers decreases the likelihood of allele drop out at the one or more loci of interest.

2. The method of claim 1, wherein the nucleic acid sample is a single cell, two cells, 3-5 cells, more than five cells, or fetal DNA isolated from maternal blood.

3. The method of claim 1, wherein the amplification is performed using a Whole Genome Amplification (WGA) kit or a Multiple Displacement Amplification (MDA) kit.

4. The method of claim 1, wherein the one or more loci of interest is a single locus of interest, 2-5 loci of interest, 5-10 loci of interest or more than 10 loci of interest.

5. The method of claim 1, further comprising synthesizing the spike-in primers.

6. The method of claim 1, wherein the spike-in primer is designed to amplify a product between about 200 bp and about 1000 bp, or between about 200 bp and about 600 bp.

7. The method of claim 1, further comprising measuring a genotype of the amplified nucleic acid sample.

8. The method of claim 1, wherein the method is used in combination with an informatics method such as the Parental Support method.

9. The method of claim 1, wherein the likelihood of allele drop out is decreased by up to about 20%, up to about 30%, up to about 40%, up to about 50%, up to about 60%, or over about 60%.

10. A method for determining the haplotype of a DNA sample, the method comprising:
dividing a DNA sample from one or more cells into a plurality of fractions;
genotyping the DNA sample in each fraction individually; and
reconstructing the haplotype of the one or more cells based on the genotyping.

11. The method of claim 10, wherein the DNA sample contains DNA from a single cell, from two cells, or from three to five cells.

12. The method of claim 10, wherein the DNA sample is divided into from two to five fractions, from six to ten fractions, from eleven to twenty fractions, from twenty-one to fifty fractions, or over fifty fractions.

13. The method of claim 10, wherein the DNA sample is not purified.

14. (canceled)

15. The method of claim 10, wherein the DNA sample originates from one or more recently lysed cells.

16. (canceled)

17. The method of claim 10, further comprising diluting the DNA sample before dividing it into fractions.

18. (canceled)

19. (canceled)

20. A method for determining the ploidy state of one or more chromosomes of a single cell containing nuclear DNA, wherein a given chromosome is associated with a known set of alleles, the method comprising:
dividing a DNA sample into a plurality of fractions;
genotyping the DNA sample in each of the fractions;
determining the number of fractions in which one or more alleles associated with a given chromosome is detected; and
using data from the determining step to determine a ploidy state of the given chromosome.

21. The method of claim 20, wherein the DNA sample is divided into from two to five fractions, from six to ten fractions, from ten to twenty fractions, or over twenty fractions.

22. (canceled)

23. The method of claim 20, further comprising amplifying the DNA sample in each of the fractions before the DNA sample is genotyped.

24. The method of claim 20, further comprising diluting the DNA sample before dividing the DNA sample into fractions.

25. (canceled)

26. (canceled)

27. The method of claim 20, wherein a Bayesian method is used to determine the most likely ploidy state.

28. The method of claim 20, wherein the Well Frequency Distribution (WFD) or the Highest Well Frequency (HWF) method is used to determine the ploidy state.

29. The method of claim 1, wherein the ploidy state, haplotype or whole genome amplification information is used for the purpose of embryo selection during in-vitro fertilization or prenatal genetic diagnosis.

30. (canceled)

31. (canceled)

32. (canceled)

33. The method of claim 10, wherein the ploidy state, haplotype or whole genome amplification information is used for the purpose of embryo selection during in-vitro fertilization or prenatal genetic diagnosis.

34. The method of claim 20, wherein the ploidy state, haplotype or whole genome amplification information is used for the purpose of embryo selection during in-vitro fertilization or prenatal genetic diagnosis.


SEQUENCE LISTING (24 sequences)

SEQ ID NO: 1 - DNA, 23 bp, Artificial Sequence
  Primer
ttctctgtgcctctgttttcttg

SEQ ID NO: 2 - DNA, 21 bp, Artificial Sequence
  Primer
tagctcatgtccctgcctctc

SEQ ID NO: 3 - DNA, 20 bp, Artificial Sequence
  Primer
tacttgtcctgctgcgatgt

SEQ ID NO: 4 - DNA, 21 bp, Artificial Sequence
  Primer
ttccccaggacattaagagcg

SEQ ID NO: 5 - DNA, 21 bp, Artificial Sequence
  Primer
tttgtttgttttgctgcttgc

SEQ ID NO: 6 - DNA, 21 bp, Artificial Sequence
  Primer
cctctccctccaactccaatt

SEQ ID NO: 7 - DNA, 21 bp, Artificial Sequence
  Primer
aggaatttggttgtggagagc

SEQ ID NO: 8 - DNA, 20 bp, Artificial Sequence
  Primer
acctgtcttccctcccagtt

SEQ ID NO: 9 - DNA, 21 bp, Artificial Sequence
  Primer
atgtggccacttgaaaaagaa

SEQ ID NO: 10 - DNA, 21 bp, Artificial Sequence
  Primer
acccacccctagaggttctga

SEQ ID NO: 11 - DNA, 21 bp, Artificial Sequence
  Primer
tgttccaggcactgaacaaca

SEQ ID NO: 12 - DNA, 21 bp, Artificial Sequence
  Primer
aacgctgtgccagattctctt

SEQ ID NO: 13 - DNA, 23 bp, Artificial Sequence
  Primer
ttaccaccttgcacacattcatt

SEQ ID NO: 14 - DNA, 23 bp, Artificial Sequence
  Primer
aaattccccaggacattaagagc

SEQ ID NO: 15 - DNA, 23 bp, Artificial Sequence
  Primer
tgctttcaaactgttgacattcc

SEQ ID NO: 16 - DNA, 23 bp, Artificial Sequence
  Primer
ggtgagtttggagacctcaaatg

SEQ ID NO: 17 - DNA, 52 bp, Artificial Sequence
  Primer
actagatctacgtgtaagtcatggacttcggttggttaatctgtgctccagt

SEQ ID NO: 18 - DNA, 52 bp, Artificial Sequence
  Primer
gaagtccatgacttacacgtagatctagtaaattccccaggacattaagagc

SEQ ID NO: 19 - DNA, 52 bp, Artificial Sequence
  Primer
actagatctacgtgtaagtcatggacttctgctttcaaactgttgacattcc

SEQ ID NO: 20 - DNA, 52 bp, Artificial Sequence
  Primer
gaagtccatgacttacacgtagatctagtagaatcatcttttgctccagagg

SEQ ID NO: 21 - DNA, 34 bp, Artificial Sequence
  Primer
agttcaacattaaataatggaaataactttctac

SEQ ID NO: 22 - DNA, 57 bp, Artificial Sequence
  Primer
actagatctacgtgtaagtcatggacttcatataacatcaattaatatgagaatcat

SEQ ID NO: 23 - DNA, 48 bp, Artificial Sequence
  Primer
gaagtccatgacttacacgtagatctagttcaagcaattctcccacgt

SEQ ID NO: 24 - DNA, 28 bp, Artificial Sequence
  Primer
cctttacatactgattttaaaactttgt